(19) United States
(12) Patent Application Publication
(10) Pub. No.: US 2024/0304317 A1
(43) Pub. Date: Sep. 12, 2024

(54) LOCATION-BASED NEUROSTIMULATION PROGRAMMING AND USER INTERACTIONS

(71) Applicant: Boston Scientific Neuromodulation Corporation, Valencia, CA (US)

(72) Inventors: Lisa Denise Moore, Glendale, CA (US); Bradley Lawrence Hershey, Carrollton, TX (US)

(21) Appl. No.: 18/590,631

(22) Filed: Feb. 28, 2024

Related U.S. Application Data
(60) Provisional application No. 63/451,013, filed on Mar. 9, 2023.

(51) Int. Cl. G16H 40/40; A61N 1/36; G16H 10/20

(52) U.S. Cl. CPC G16H 40/40; A61N 1/3605; G16H 10/20

(57) ABSTRACT

A system to analyze patient location activity in connection with a neurostimulation treatment may: receive tagged location data associated with a patient undergoing the neurostimulation treatment, including tagged location data provided from a patient computing device that indicates a visit of the patient to one or more locations; identify a location type of the locations, using the tagged location data; determine characteristics of the visit at the location type, using the tagged location data; and control a workflow related to the neurostimulation treatment, based on the characteristics of the visit of the patient at the location type. In further examples, such workflows may include a patient interaction workflow, a caregiver workflow, or a clinician workflow, to generate tasks or provide informational alerts based on the patient location activity. Further examples are provided for closed-loop programming of a neurostimulation device based on the patient location activity.

### CLAIM OF PRIORITY

This application claims the benefit of U.S. Provisional Application No. 63/451,013 filed on Mar. 9, 2023, which is hereby incorporated by reference in its entirety.

### TECHNICAL FIELD

This document relates generally to data processing obtained in connection with the use of medical devices, and more particularly, to systems, devices, and methods for tracking location data in connection with patient uses of implanted electrical stimulation, including automatically collected and user-controlled location information during neurostimulation treatments used for pain treatment, movement disorders, and/or management of such conditions.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). A neurostimulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system that uses similar pulses of electricity at particular locations in the brain to reduce symptoms of essential tremors, Parkinson's disease, psychological disorders, or the like.

Various approaches are being developed to enable personalized programming and optimized forms of programming used by neurostimulation systems, including partially or fully automated forms of generating or delivering specific neurostimulation parameters known as closed-loop programming. Some closed-loop programming approaches can, for example, customize changes to stimulator programs, such as to suggest or automatically select a program when a particular condition is identified. However, personalized and closed-loop programming typically requires a robust and extensive set of data inputs, to accurately learn characteristics about the individual patient and to adapt to the user's particular behavior. Although there has been a progressive increase in digital connectivity and approaches to track user behavior and collect user data, there are a lack of tools to understand why user behavior occurs and how to better customize data operations (programming, questionnaires, workflows) to a particular patient. In particular, most approaches for tracking user behavior do not consider the location of the patient, and the health, social, or travel characteristics of the patient when as they visit different locations.

### SUMMARY

An example (e.g., “Example 1”) is a system to analyze patient location activity in connection with a neurostimulation treatment, the system comprising: one or more processors; and one or more memory devices comprising instructions, which when executed by the one or more processors, cause the one or more processors to: receive tagged location data associated with a patient undergoing the neurostimulation treatment, wherein the tagged location data is provided from a patient computing device, wherein the tagged location data indicates a visit of the patient to one or more locations; identify a location type of the one or more locations, using the tagged location data; determine characteristics of the visit of the patient at the location type, using the tagged location data; and control a workflow related to the neurostimulation treatment, based on the characteristics of the visit of the patient at the location type.

In Example 2, the subject matter of Example 1 optionally includes subject matter where the tagged location data includes an identifier of the location type, wherein the location type is one of a plurality of location types defined in the system, and wherein the tagged location data provided from the patient computing device to the system does not include an identifiable geographic location of the one or more locations.

In Example 3, the subject matter of Example 2 optionally includes subject matter where the identifier of the location type is automatically provided by the patient computing device, in response to the patient computing device having detected the visit of the patient to one or more geographic locations associated with the location type.

In Example 4, the subject matter of Example 3 optionally includes subject matter where the location type is a type of medical care location, and wherein the location type is categorized as one of a: hospital, clinic, rehabilitation facility, nursing home facility, programming location, or pharmacy, associated with one or more medical professional or service in connection with the neurostimulation treatment.

In Example 5, the subject matter of any one or more of Examples 2-4 optionally includes subject matter where the identifier of the location type is provided by the patient computing device, in response to selection of the location type by a user of the patient computing device.

In Example 6, the subject matter of any one or more of Examples 1-5 optionally includes subject matter where the tagged location data includes an identifier associated with the patient or the patient computing device.

In Example 7, the subject matter of any one or more of Examples 1-6 optionally includes subject matter where the workflow related to the neurostimulation treatment is a patient interaction workflow to occur with the patient, and wherein the instructions further cause the one or more processors to: generate one or more questionnaires or interaction tasks to provide to the patient based on the characteristics of the visit of the patient at the location type.

In Example 8, the subject matter of any one or more of Examples 1-7 optionally includes subject matter where the workflow related to the neurostimulation treatment is a caregiver workflow to occur with a caregiver or a medical device company representative associated with the patient, and wherein the instructions further cause the one or more processors to: generate one or more alerts to at least one of the caregiver or a medical device company representative based on the characteristics of the visit of the patient at the location type.

In Example 9, the subject matter of any one or more of Examples 1-8 optionally includes subject matter where the workflow related to the neurostimulation treatment is a clinician workflow to occur with a clinician or a medical device company representative associated with the patient, and wherein the instructions further cause the one or more processors to: generate one or more alerts to at least one of the clinician or the medical device company representative based on the characteristics of the visit of the patient at the location type.

In Example 10, the subject matter of any one or more of Examples 1-9 optionally includes subject matter where the characteristics of the visit of the patient are determined from one or more of: weather event data associated with weather at the one or more locations; duration event data associated with a duration of the visit at the one or more locations; travel event data associated with travel of the patient to or from the one or more locations; purpose event data associated with a purpose of the visit at the one or more locations; or social interaction event data associated with interactions occurring between the patient and one or more persons at the one or more locations.

In Example 11, the subject matter of any one or more of Examples 1-10 optionally includes subject matter where the instructions further cause the one or more processors to: determine usage of a neurostimulation program by a neurostimulation device at the location type; and identify a patient state based on the usage of the neurostimulation program and the characteristics of the visit of the patient at the location type.

In Example 12, the subject matter of Example 11 optionally includes subject matter where the workflow related to the neurostimulation treatment provides a control to a closed-loop programming therapy of the neurostimulation device based on the identified patient state, wherein the patient state relates to one or more of: sleep, actigraphy, accelerometry, pain, movement, stress, disease-related symptoms, emotional state, medication state, or activity during use of the neurostimulation program.

In Example 13, the subject matter of Example 12 optionally includes subject matter where the closed-loop programming therapy causes an automatic change to neurostimulation programming settings on the neurostimulation device, and wherein the automatic change to the neurostimulation programming settings controls one or more of: pulse patterns, pulse shapes, a spatial location of pulses, electric fields or activating functions of active electrodes, waveform shapes, or a spatial location of waveform shapes, of for modulated energy provided with a plurality of leads of the neurostimulation device.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a device to analyze patient location activity in connection with a neurostimulation treatment, the device comprising: one or more processors; and one or more memory devices comprising instructions, which when executed by the one or more processors, cause the one or more processors to: receive tagged location data associated with a patient undergoing the neurostimulation treatment, wherein the tagged location data is provided from a patient computing device, wherein the tagged location data indicates a visit of the patient to one or more locations; identify a location type of the one or more locations, using the tagged location data; determine characteristics of the visit of the patient at the location type, using the tagged location data; and control a workflow related to the neurostimulation treatment, based on the characteristics of the visit of the patient at the location type.

In Example 17, the subject matter of Example 16 optionally includes subject matter where the tagged location data includes an identifier of the location type, wherein the location type is one of a plurality of defined location types, and wherein the tagged location data provided from the patient computing device does not include an identifiable geographic location of the one or more locations.

In Example 18, the subject matter of Example 17 optionally includes subject matter where the identifier of the location type is automatically provided by the patient computing device, in response to the patient computing device having detected the visit of the patient to one or more geographic locations associated with the location type.

In Example 19, the subject matter of Example 18 optionally includes subject matter where the location type is a type of medical care location, wherein the location type is categorized as one of a: hospital, clinic, rehabilitation facility, nursing home facility, programming location, or pharmacy, associated with one or more medical professional or service in connection with the neurostimulation treatment; and wherein the identifier of the location type is provided by the patient computing device, in response to selection of the location type by a user of the patient computing device.

In Example 20, the subject matter of any one or more of Examples 17-19 optionally includes subject matter where the tagged location data includes an identifier associated with the patient or the patient computing device.

In Example 21, the subject matter of any one or more of Examples 16-20 optionally includes subject matter where the workflow related to the neurostimulation treatment is a patient interaction workflow to occur with the patient, and wherein the instructions further cause the one or more processors to: generate one or more questionnaires or interaction tasks to provide to the patient based on the characteristics of the visit of the patient at the location type.

In Example 22, the subject matter of any one or more of Examples 16-21 optionally includes subject matter where the workflow related to the neurostimulation treatment is a caregiver workflow to occur with a caregiver or a medical device company representative associated with the patient, and wherein the instructions further cause the one or more processors to: generate one or more alerts to at least one of the caregiver or a medical device company representative based on the characteristics of the visit of the patient at the location type.

In Example 23, the subject matter of any one or more of Examples 16-22 optionally includes subject matter where the workflow related to the neurostimulation treatment is a clinician workflow to occur with a clinician or a medical device company representative associated with the patient, and wherein the instructions further cause the one or more processors to: generate one or more alerts to at least one of the clinician or the medical device company representative based on the characteristics of the visit of the patient at the location type.

In Example 24, the subject matter of any one or more of Examples 16-23 optionally includes subject matter where the characteristics of the visit of the patient are determined from one or more of: weather event data associated with weather at the one or more locations; duration event data associated with a duration of the visit at the one or more locations; travel event data associated with travel of the patient to or from the one or more locations; purpose event data associated with a purpose of the visit at the one or more locations; or social interaction event data associated with interactions occurring between the patient and one or more persons at the one or more locations.

In Example 25, the subject matter of any one or more of Examples 16-24 optionally includes subject matter where the instructions further cause the one or more processors to: determine usage of a neurostimulation program by a neurostimulation device at the location type; and identify a patient state based on the usage of the neurostimulation program and the characteristics of the visit of the patient at the location type; wherein the workflow related to the neurostimulation treatment provides a control to a closed-loop programming therapy of the neurostimulation device based on the identified patient state, and wherein the patient state relates to one or more of: sleep, actigraphy, accelerometry, pain, movement, stress, disease-related symptoms, emotional state, medication state, or activity during use of the neurostimulation program.

Example 26 is a method for analyzing patient location activity in connection with a neurostimulation treatment, comprising: receiving tagged location data associated with a patient undergoing the neurostimulation treatment, wherein the tagged location data is provided from a patient computing device, wherein the tagged location data indicates a visit of the patient to one or more locations; identifying a location type of the one or more locations, using the tagged location data; determining characteristics of the visit of the patient at the location type, using the tagged location data; and controlling a workflow related to the neurostimulation treatment, based on the characteristics of the visit of the patient at the location type.

In Example 27, the subject matter of Example 26 optionally includes subject matter where the tagged location data includes an identifier of the location type, wherein the location type is one of a plurality of defined location types, and wherein the tagged location data provided from the patient computing device does not include an identifiable geographic location of the one or more locations.

In Example 28, the subject matter of Example 27 optionally includes subject matter where the identifier of the location type is automatically provided by the patient computing device, in response to the patient computing device having detected the visit of the patient to one or more geographic locations associated with the location type.

In Example 29, the subject matter of Example 28 optionally includes subject matter where the location type is a type of medical care location, wherein the location type is categorized as one of a: hospital, clinic, rehabilitation facility, nursing home facility, programming location, or pharmacy, associated with one or more medical professional or service in connection with the neurostimulation treatment; and wherein the identifier of the location type is provided by the patient computing device, in response to selection of the location type by a user of the patient computing device.

In Example 30, the subject matter of any one or more of Examples 27-29 optionally includes subject matter where the tagged location data includes an identifier associated with the patient or the patient computing device.

In Example 31, the subject matter of any one or more of Examples 26-30 optionally includes subject matter where the workflow related to the neurostimulation treatment is a patient interaction workflow to occur with the patient, and wherein the method further comprises: generating one or more questionnaires or interaction tasks to provide to the patient based on the characteristics of the visit of the patient at the location type.

In Example 32, the subject matter of any one or more of Examples 26-31 optionally includes subject matter where the workflow related to the neurostimulation treatment is a caregiver workflow to occur with a caregiver or a medical device company representative associated with the patient, and wherein the method further comprises: generating one or more alerts to at least one of the caregiver or a medical device company representative based on the characteristics of the visit of the patient at the location type.

In Example 33, the subject matter of any one or more of Examples 26-32 optionally includes subject matter where the workflow related to the neurostimulation treatment is a clinician workflow to occur with a clinician or a medical device company representative associated with the patient, and wherein the method further comprises: generating one or more alerts to at least one of the clinician or the medical device company representative based on the characteristics of the visit of the patient at the location type.

In Example 34, the subject matter of any one or more of Examples 26-33 optionally includes subject matter where the characteristics of the visit of the patient are determined from one or more of: weather event data associated with weather at the one or more locations; duration event data associated with a duration of the visit at the one or more locations; travel event data associated with travel of the patient to or from the one or more locations; purpose event data associated with a purpose of the visit at the one or more locations; or social interaction event data associated with interactions occurring between the patient and one or more persons at the one or more locations.

In Example 35, the subject matter of any one or more of Examples 26-34 optionally include determining usage of a neurostimulation program by a neurostimulation device at the location type; and identifying a patient state based on the usage of the neurostimulation program and the characteristics of the visit of the patient at the location type; wherein the workflow related to the neurostimulation treatment provides a control to a closed-loop programming therapy of the neurostimulation device based on the identified patient state, and wherein the patient state relates to one or more of: sleep, actigraphy, accelerometry, pain, movement, stress, disease-related symptoms, emotional state, medication state, or activity during use of the neurostimulation program.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a data processing system to collect and analyze healthcare-related data in connection with the use of neurostimulation programs and closed-loop neurostimulation programming.

FIG. 2 illustrates a neuromodulation system as an example of a medical device system, according to an example.

FIG. 3 further illustrates a neuromodulation system as an ambulatory medical device, such as implemented by a neurostimulation treatment, according to an example.

FIG. 4 illustrates a neuromodulation device connected to a programming device, according to an example.

FIG. 5 illustrates data interactions with a data processing system for operation of a neuromodulation device with location-assisted programming and location-based workflows, according to an example.

FIG. 6 illustrates data types and processing logic associated with data input or output for location-based data processing, according to an example.

FIG. 7A depicts a flowchart of workflow operations for deriving purpose of a user visit to a location, according to an example.

FIG. 7B depicts a flowchart of workflow operations for deriving social interaction of a user visit to a location, according to an example.

FIG. 7C depicts a flowchart of workflow operations for targeting tasks to a patient based on location data, according to an example.

FIG. 7D depicts a flowchart of workflow operations for targeting tasks and information to caregivers based on patient location data, according to an example.

FIG. 7E depicts a flowchart of workflow operations for informing physicians or other medical professionals based on patient location data, according to an example.

FIG. 8A and FIG. 8B depict data input user interfaces that include functionality to enable tagging of locations and location types, according to an example.

FIG. 9A and FIG. 9B depict data input user interfaces that include functionality to receive additional information and user inputs, according to an example.

FIG. 10 illustrates a data processing flow for controlling the neurostimulation treatment of a human patient based on location-based data processing operations, according to an example.

FIG. 11 illustrates a processing method implemented by a system or device to analyze patient location activity in connection with a neurostimulation treatment programming, according to an example.

FIG. 12 illustrates a block diagram of a system (e.g., a computing system) for performing analysis of patient location data, according to an example.

FIG. 13 illustrates a block diagram of an embodiment of a system (e.g., a computing system) implementing neurostimulation programming circuitry to cause programming of an implantable electrical neurostimulation device, according to an example.

FIG. 14 is a block diagram illustrating a machine in the example form of a computer system, within which a set or sequence of instructions may be executed to cause the machine to perform any one of the methodologies discussed herein, according to an example.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments of the present subject matter relate to user interfaces, data communications, data processing operations, location tracking methods and systems, and computing systems and devices used in connection with a neurostimulation programming system, neurostimulation devices, and associated data processing systems. As an example, aspects refer to user interface screens and software-initiated processes that collect (or, cause the collection of) relevant location data associated with user behavior and activity. The location data, once collected, can be used to determine a patient state at a particular location, and to automatically derive a purpose of what the patient is doing at the particular location. Consequently, this location data can be used to effect a neurostimulation treatment workflow, including to drive new interaction questions and recommendations, to produce new reprogramming changes, to cause alerts or notifications, or to effect a variety of other operations associated with neurostimulation programming.

Location data can be used to understand where a patient is, what activity the patient is doing or might be doing at the location, what other persons are around the patient, and what types of social interactions are occurring with the patient. Such location data can also be analyzed to better understand a patient state, which can then be used to trigger additional user interactions such as questionnaires that are customized to collect details on what the patient was experiencing at the location. Although some prior approaches have attempted to monitor patient activity and behavior during neurostimulation treatment, it is often not apparent why a patient's activity and behavior may have changed. The following proposes systems and methods to use location data to analyze a patient's activity and behavior, to reduce user interaction burden, inform data presentation, and ultimately attempt improvements in neurostimulation programming and treatment.

In an example, a user interface is provided in a smartphone app to allow the tagging of non-specific geolocation data to one or more particular locations. As used herein, a “geolocation” refers to an area or multiple areas determined from digital tracking information, which may correspond to a location measurement or set of measurements taken with a sensor or device (e.g., a GPS coordinate, a building identified by a mapping service, a geofenced area defined by a phone platform, etc.). Geolocation information may be used to associate a location (or multiple locations) with a particular meaning, such as “Home”, “Doctor's Office”, “Pharmacy”, “Airport”, etc. This may be accomplished with the use of “data tags” which associate location data with properties such as the general location, the location type, and a unique identifier of the location or the location type. Geolocation information may be communicated to a data processing service that obtains a larger view of the patient activity and activity from among multiple patients.

Various forms of data processing may occur based on the collected location data, including recommendations, alerts, reprogramming or device operation workflows, and data-driven activities. As one example, location data may be used to generate targeted questions about what the user was doing at the location type, to understand user behavior and activity and to reduce the amount of time the patient needs to answer background questions. Also, such data can be used to improve or enhance a closed-loop programming system, to automatically select or recommend therapies from new programming of a neurostimulation device based on the observed behavior and activity at a location type. Accordingly, the presently disclosed data processing can lead to a variety of optimizations in the use and deployment of new programs, modification of existing programs, optimization of program usage and schedules, and other neurostimulation device improvements.

The following describes a “data collection platform”, “data processing system”, and “data service” that generally refers to portions of a computer platform (also referred to as a “compute platform” provided by, e.g., a combination of hardware, firmware and software) with a set of capabilities for collecting, processing, and generating location data in connection with user activity, neurostimulation programming, and the user interfaces discussed below. A computer platform may be a single platform (e.g., at a single cloud service) or may be organized as more than one platform (e.g., among multiple distributed computing locations) that are configured to cooperate with each other. A computer platform may obtain data from one patient device or from among more than one patient device. Thus, for example, a therapy device such as an implanted neuromodulation device may provide some portion of the collected data, and a user device (e.g., smartphone) with an interactive user interface (e.g., provided by a smartphone app) and a location sensor may provide another portion of the collected data. A computer platform may also obtain data from other sensor(s) and other data source(s).

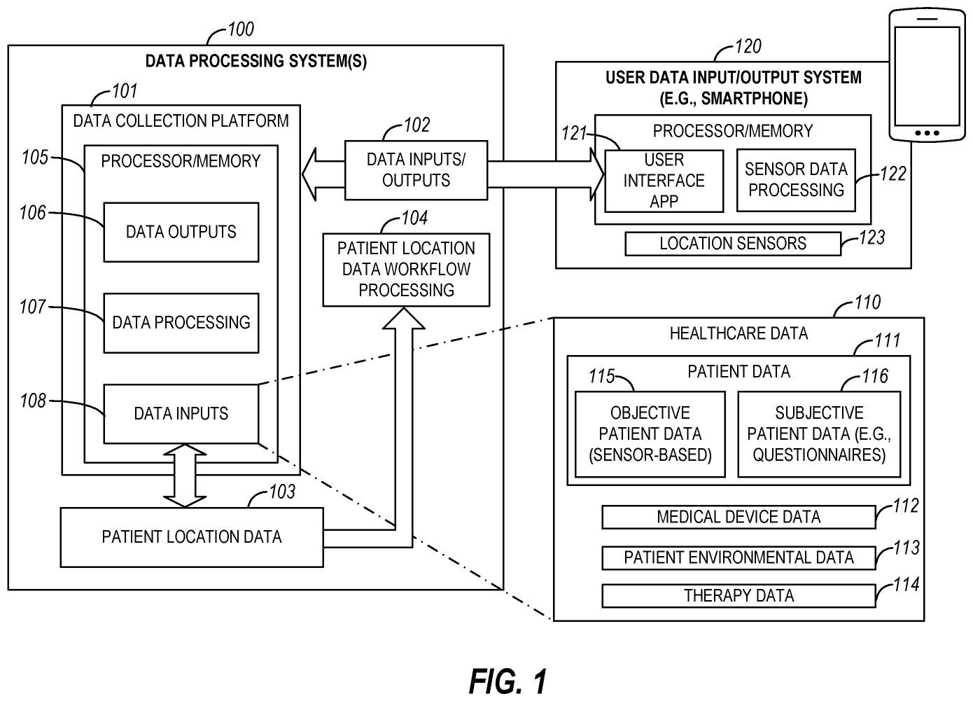

FIG. 1 illustrates, by way of example, an embodiment of a data processing system 100 configured to collect and analyze healthcare-related data in connection with the use of neurostimulation programs and location-based workflows. The illustrated data processing system 100 is configured to include at least one data collection platform 101, which operates to collect and provide data inputs/outputs 102. The data collection platform 101 is configured to process (e.g., filter, extract, transform) the input data to produce patient location data 103, which then can be used for one or more iterations of patient location data workflow processing 104. The data collection platform 101 can also generate output data in the form of recommendations, suggestions, questionnaires, and other information based on the patient location data workflow processing 104 or the patient location data 103.

The data processing system 100 may be implemented at one or more server(s) or other systems remotely located from the patient. The data processing system 100 may use various network protocols to communicate and transfer data through one or more networks which may include the Internet. The data processing system 100 and data collection platform 101 may include at least one processor configured to execute instructions stored in memory (e.g., depicted as processor(s)/memory 105) to generate or evaluate data outputs 106, to obtain or evaluate data inputs 108, and to perform data processing 107 on both inputs and outputs and accompanying location data. For instance, the data inputs 108 may be processed to derive or associate additional aspects (e.g., context, tags, metadata) of the patient location data 103, such as based on the receipt of additional data (e.g., custom geolocation tags) provided within the user interface app 121.

The data inputs 108 may include location or contextual information obtained directly or indirectly from the patient, or such data inputs 108 may relate to healthcare data 110 associated with the patient or the treatment. Examples of healthcare data 110 may include patient data 111, medical device data 112, patient environmental data 113, therapy data 114, or various combinations thereof. The patient data 111 may include objective data 115 such as data collected from physiological sensor(s) and subjective data 116 such as data collected from user-answered question(s) (e.g., “How do you rate your pain?”, “What prescriptions did you refill at the pharmacy?”). The objective data 115 and subjective data 116 may include or be associated with location data or other location-based characteristics.

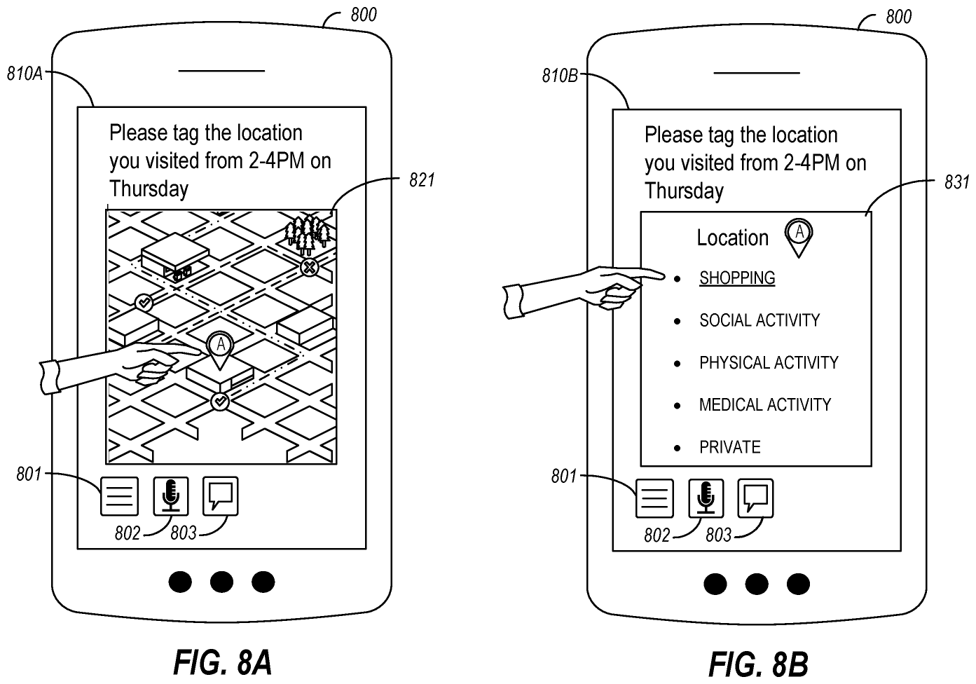

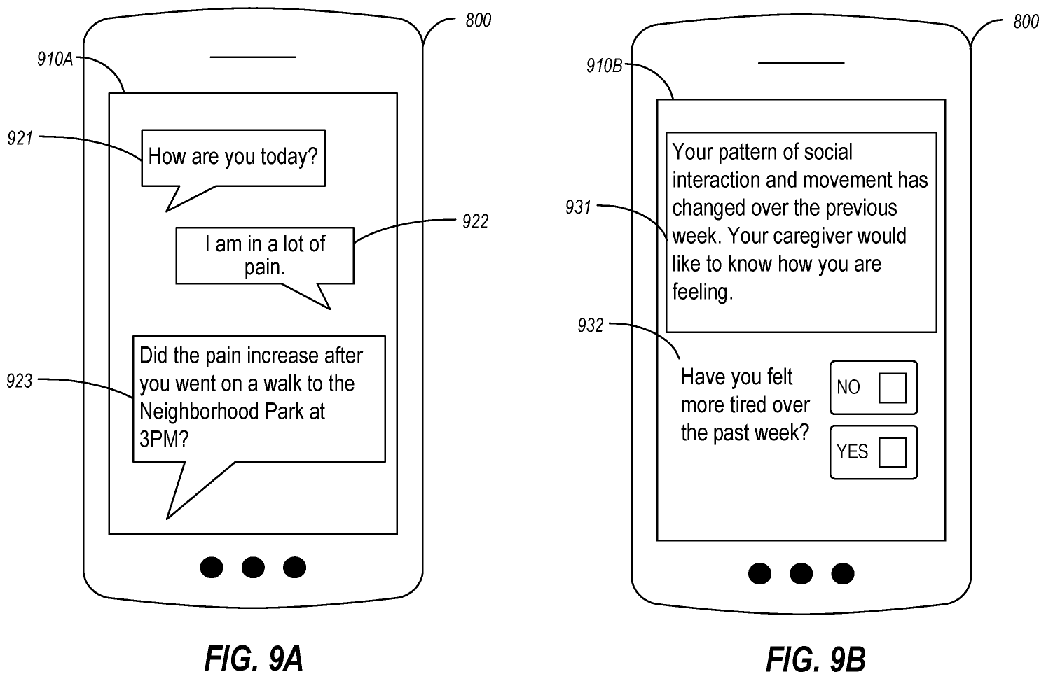

The user data input/output system 120 may be implemented at one or more devices located at or operated by the patient, such as via a smartphone, personal computer, tablet, smart home device, a remote programmer, a programming device, or another computer device or platform capable of collecting input and providing output. The user data input/output system 120 may include at least one processor configured to execute instructions stored in memory (e.g., depicted as processor(s)/memory 105) to provide or generate the data input(s) and output(s) 102 related to healthcare data and location information. One such example is a user interface application 121 implemented as a graphical user interface (GUI). Examples of GUIs for data inputs and outputs include smartphone apps that are illustrated in FIGS. 8A, 8B, 9A, and 9B, discussed below. Another example is sensor data processing 122 which can be used to determine the patient state (e.g., amount of activity, duration of sleep) at a particular time or location.

The user data input/output system 120 may include or interface with one or more location sensors 123 (e.g., global positioning system (GPS) sensors) or location functionality to track, identify, determine, or derive a geographic location of the patient (and ultimately, a location type of this geographic location). Other sensors such as physiological sensors may also be provided by the user data input/output system 120, to capture measurements directly or indirectly from the patient or from associated external systems. Examples of external system(s) include remote controls, programmers, phones, tablets, smart watches, personal computers, and the like. Thus, the user data input/output system 120 may be configured to provide all or portions of the medical device data 112, the patient environmental data 113, or the therapy data 114.

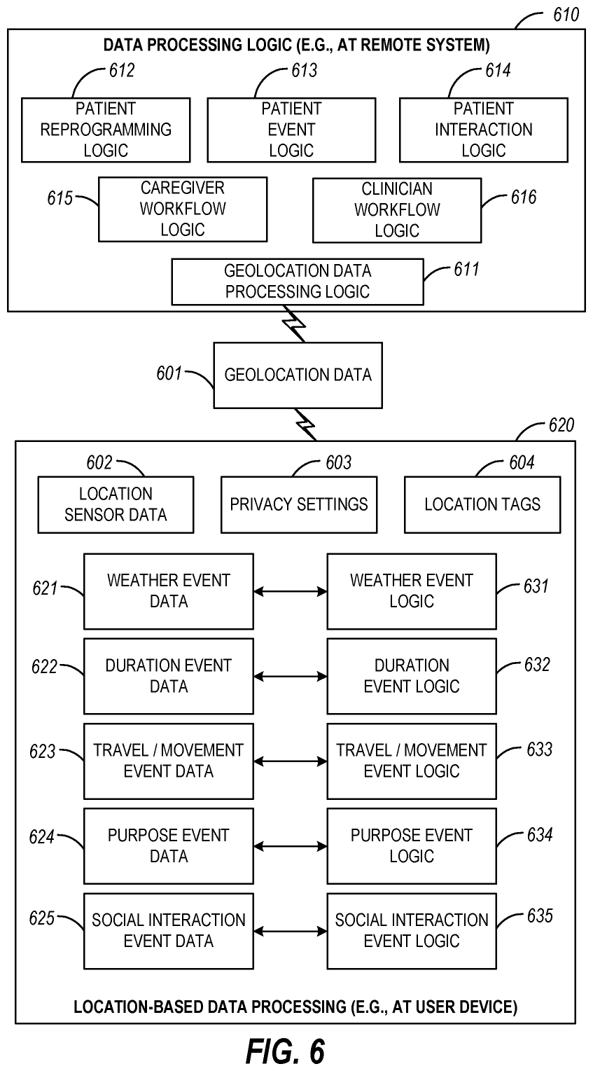

The patient location data workflow processing 104 operates to identify, trigger, or cause workflow actions based on the patient location data 103, including which location(s) the patient has visited, what activities that the patient performed at the location(s), and the like. The patient location data workflow processing 104 may consider other aspects of patient-specific or population data such as the healthcare data 110, sensor data, and rules and information from a variety of data sources. More details of location data types and location-based data processing is discussed with reference to FIG. 6, below.

In some examples, the data collection platform 101 and the data processing system 100 may directly interface with one or more medical device(s), external system(s) or other healthcare related data source(s) to collect the healthcare data 110. One or more of the medical device(s), external system(s) or other healthcare-related data source(s) may include technology used by the data processing system 100 to collect data, and thus may form part of the data collection platform 101. Examples of medical devices include implantable and wearable devices. The medical device may be configured to only collect data, to only deliver therapy, or to both collect data and deliver therapy. The medical device may be configured to collect and provide medical device data such as device model, configuration, settings, and the like. Thus, the medical device may provide the patient data 111, the medical device data 112, the environmental data 113, and therapy data 114, particularly in specialized neurostimulation programming environments.

Other healthcare-related data source(s) may include patient data received via a provider's server that stores patient health records. For example, patients may use a patient portal to access their health records such as test results, doctor notes, prescriptions, and the like. Other healthcare-related data sources may include apps on a patient's smartphone or other computing device, or the data on a server accessed by those apps. By way of example and not limitation, this type of data may include heart rate, blood pressure, weight, and the like collected by the patient in their home. In another example, an app on a phone or patient's device may include or may be configured to access environmental data such as weather data and air quality information or location elevation data such as may be determined using cellular networks and/or GPS technologies. Weather data may include, but is not limited to, barometric pressure, temperature, sunny or cloud cover, wind speed, and the like. These and other examples of patient environmental data 113 may be correlated to information in the patient location data 103, so that analysis can be performed of what environment the patient was encountering at a particular location.

The data inputs/outputs 102 may be provided with data transferred via at least one network. The data transfer may use various network protocols to communicate and transfer data through one or more networks which may but does not necessarily include the Internet and/or various wireless networks, which may include short range wireless technology such as Bluetooth. The data may be transferred directly from at least one of the external systems and/or may be transferred directly from at least one of the medical device(s). Further, the external system(s) may be configured to receive data from an associated medical device(s) and/or receive data from other healthcare-related data source(s), and then transfer the data through the network(s) to the data receiving system(s).

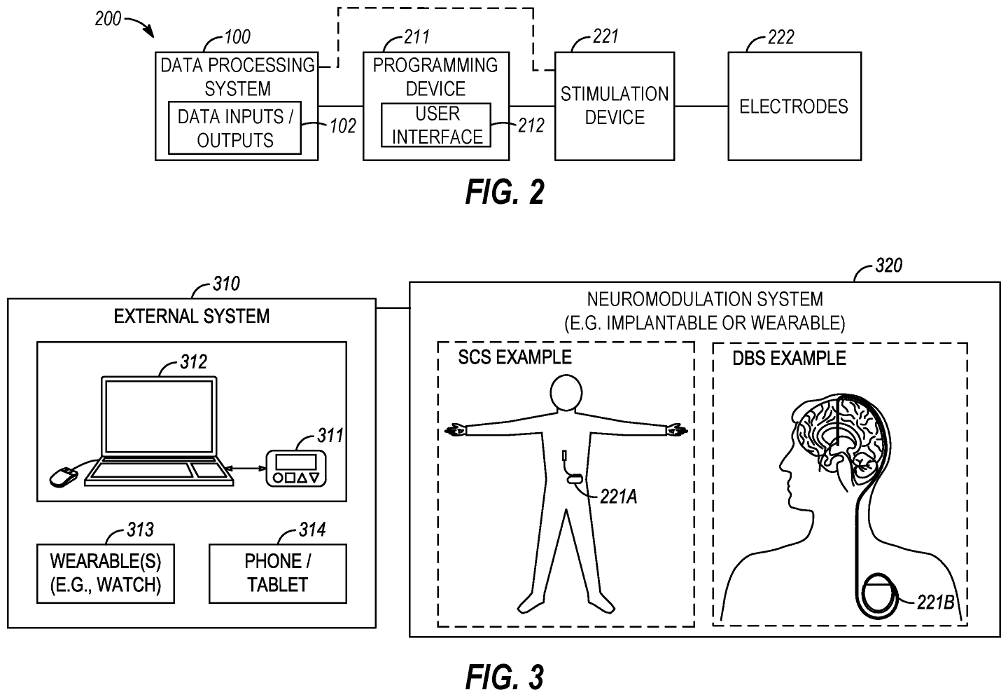

FIG. 2 illustrates a neuromodulation system as an example of a medical device system. The medical device may be configured, by way of example and not limitation, to deliver an electrical therapy using one or more electrodes 222 provided by a stimulation device 221. In the illustrated embodiment, the medical device may be a neurostimulation device, and the system may be a neuromodulation system 200.

The illustrated neuromodulation system 200 includes electrodes 222, the stimulation device 221 and a programming system such as a programming device 211. The programming system may include multiple devices. The electrodes 222 are configured to be placed on or near one or more neural targets in a patient. The stimulation device 221 is configured to be electrically connected to the electrodes 222 and deliver neuromodulation energy, such as in the form of electrical pulses, to the one or more neural targets though the electrodes 222. The system may also include sensing circuitry to sense a physiological signal, which may but does not necessarily form a part of stimulation device 221. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver, using one or more program. The programming device 211 thus provides the user with accessibility to the user-programmable parameters. The programming device 211 may also provide the use with data indicative of the sensed physiological signal or feature(s) of the sensed physiological signal.

In various embodiments, the programming device 211 is configured to be communicatively coupled to the stimulation device 221 via a wired or wireless link. In various embodiments, the programming device 211 includes a user interface 212 such as a graphical user interface (GUI) that allows the user to set and/or adjust values of the user-programmable modulation parameters. The user interface 212 may also allow the user to view the data indicative of the sensed physiological signal or feature(s) of the sensed physiological signal and may allow the user to interact with that data. The data is provided to a data processing system 100 which provides various data inputs and outputs 102 to assist the user with the operation, configuration, maintenance, or improvement of the stimulation device 221, such as for the collection of location-related data as discussed herein.

In some examples, the stimulation device 221 and the programming device 211 may contribute data that is associated with the patient location data 103 used in the data processing system 100. The user interface 212 of the programming device 211 may also allow a user to answer questions that provide location details or location-relevant data (e.g., collecting information when used at a doctor's office, or when used at home), although other devices such as a patient smartphone may be used to obtain location data and inputs as discussed below. Therapy parameters, programming selection, electrode selection, and other operational parameters may also provide healthcare-related data for use by the data processing system 100. Additional sensor(s) may also provide healthcare-related data for use by the data processing system 100.

FIG. 3 illustrates, by way of example and not limitation, the neurostimulation system of FIG. 2 implemented in a spinal cord stimulation (SCS) system or a deep brain stimulation (DBS) system. The illustrated neuromodulation system 320 connects with an external system 310 that may include at least one programming device. The illustrated external system 310 may include a clinician programmer 312 configured for use by a clinician to communicate with and program the neurostimulator, and a remote control device 311 configured for use by the patient to communicate with and program the neurostimulator. For example, the remote control device 311 may allow the patient to turn a therapy on and off and/or may allow the patient to adjust patient-programmable parameter(s) of the plurality of modulation parameters (e.g., by switching programs).

FIG. 3 further illustrates the neuromodulation system 320 as an ambulatory medical device, such as implemented by stimulation device 221A or stimulation device 221B. Examples of ambulatory devices include wearable or implantable neuromodulators. The external system 310 may include a network of computers, including computer(s) remotely located from the ambulatory medical device that are capable of communicating via one or more communication networks with the programmer 312 and/or the remote control device 311. The remotely located computer(s) and the ambulatory medical device may be configured to communicate with each other via another external device such as the programmer 312 or the remote control device 311.

The external system 310 may also include one or more wearables 313 and a portable device 314 such as a smartphone or tablet. In some examples, the wearables 313 and the portable device 314 may allow a user to obtain and provide input data, including to answer questions (e.g., on a phone/tablet screen) or to input sensor data values (e.g., from a physiologic sensor of a wearable) as part of a data collection process. In some examples, the remote control device 311 and/or the programmer 312 also may allow a user (e.g., patient, caregiver, clinician, or manufacturer/medical device company representative (“rep”)) to answer questions and provide input as part of a data collection process. The remote control device 311 and/or the programmer 312 may be used to provide other aspects of the input data, including usage data of various neurostimulation programs, events associated with such programs, location or activity events, and the like.

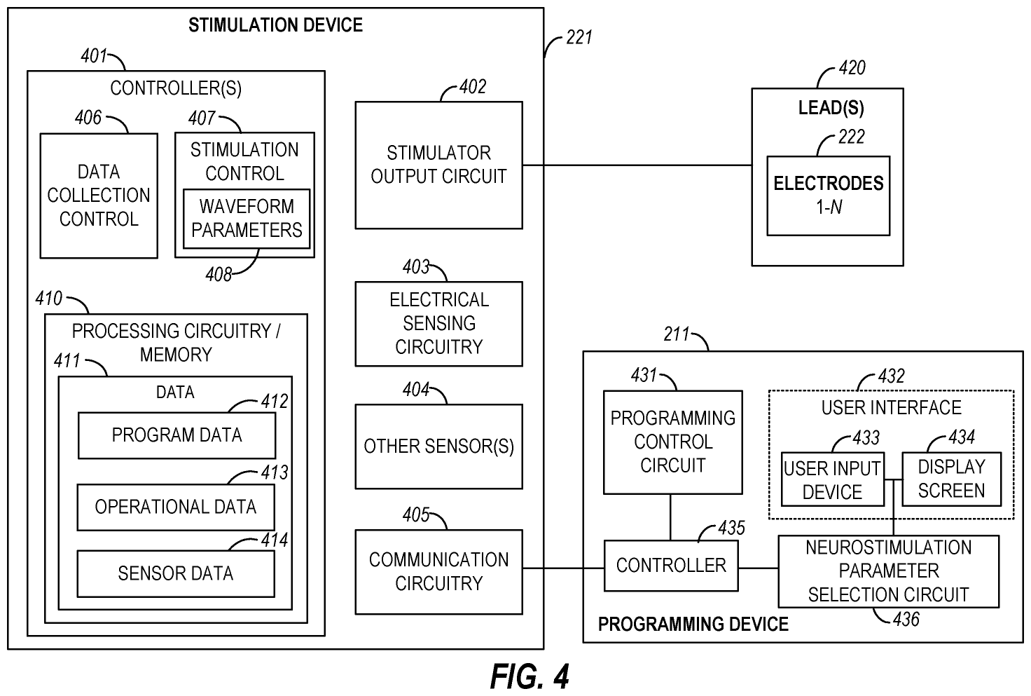

FIG. 4 illustrates, by way of example, an embodiment of a neuromodulation device, such as may be implemented as the stimulation device 221 illustrated in FIGS. 2 and 3. The stimulation device 221 may be configured to be connected to electrode(s) 222, illustrated as N electrodes, via one or more leads 420. Any one or more of the electrodes 222 may be configured for use to deliver modulation energy, sense electrical activity, or both deliver modulation energy and sense electrical activity. The stimulation device 221 may include a stimulator output circuit 402 configured to deliver modulation energy to electrode(s). The stimulator output circuit 402 may be configured with multiple (e.g., two or more) channels for delivering modulation energy, where each channel may be independently controlled with respect to other channel(s). For example, the stimulator output circuit 402 may have independent sources such as independent current sources or independent voltage sources.

In various examples, the electrodes 222 may include a stimulation electrode or a sensing electrode. The stimulation electrode is configured for use in delivering modulation energy, and the sensing electrode is configured for use in sensing electrical activity. As illustrated, the stimulation electrode may also be used in sensing electrical activity, and the sensing electrode may also be used in delivering modulation energy. Thus, the term “stimulation electrode” does not necessary exclude the electrode from also being used to sense electrical activity; and the term “sensing electrode” does not necessarily exclude the electrode from also being used to deliver modulation energy.

The stimulation device 221 may include electrical sensing circuitry 403 configured to receive sensed electrical energy from the electrode(s), such as may be used to sense electrical activity in neural tissue or muscle tissue. The sensing circuitry may be configured to process signals in multiple (e.g., two or more) channels. By way of example and not limitation, the electrical sensing circuitry 403 may be configured to amplify and filter the signal(s) in the channel(s).

The controller 401 may be configured to detect one or more features in the sensed signals. Examples of features that may be detected include peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline. Detected feature(s) may be fed into a control algorithm, which may use relationship(s) between the feature(s) and waveform parameter(s) to determine feedback for closed-loop control of the therapy. Some embodiments of the stimulation device 221 may include or be configured to receive data from other sensor(s) 404. The other sensor(s) 404 may include physiological sensor(s), environmental sensor(s), or proximity sensor(s).

The stimulation device 221 may include a controller 401 operably connected to the stimulator output circuit 402 and the electrical sensing circuitry 403. The controller 401 may include a stimulation control 407 (e.g., logic) configured for controlling the stimulator output circuit 402. For example, the stimulation control 407 may include start/stop information for the stimulation and/or may include relative timing information between stimulation channels. The stimulation control 407 may include waveform parameters 408 (e.g., associated with a program or a defined set of parameters) that control the waveform characteristics of the waveform produced by the stimulator output circuit 402. The waveform parameters 408 may include, by way of example and not limitation, amplitude, frequency, and pulse width parameters. The waveform parameters 408 may include, by way of example and not limitation, regular patterns such as patterns regularly repeat with same pulse-to-pulse interval and/or irregular patterns of pulses such as patterns with variable pulse-to-pulse intervals. The waveform parameters may, but do not necessarily, define more than one waveform shape (e.g., including a shape other than square pulses with different widths or amplitudes). The stimulation control 407 may be configured to change waveform parameter(s) (e.g., one or more waveform parameters) in response to user input and/or automatically in response to feedback.

The controller 401 may include a data collection control 406 configured for use by the stimulation device 221, and the data collection platform 101 of a data processing system 100 (see FIGS. 1-2), to collect healthcare related data. The controller 401 may include a processor and/or memory 410 (e.g., with instructions) for use to control the data collection using the data collection control 406 and control the stimulation via the stimulation control 407. The memory may also provide storage for storing different types of collected healthcare-related data 411, such as program data 412, operational data 413, sensor data 414, and the like. Examples of sensor data 414 collected by the stimulation device 221 or other devices discussed herein may include, by way of example and not limitation, heart rate, heart rate variability, oxygen saturation, activity, posture, steps, gait, temperature, evoked compound action potentials (ECAPS), electromyograms (EMGs), electroencephalograms (EEGs), weight, blood pressure, and the like. Examples of program data 412 may include, by way of example and not limitation, stimulation settings such as amplitude, pulse width, pulse frequency period, duration of burst of pulses, active electrodes, electrode fractionalization controlling the distribution of energy (e.g., current) to active electrodes, waveforms, pulse patterns including various complex patterns, and the like. Examples of operational data 413 of the stimulation device 221 may include, by way of example and not limitation, electrode-tissue impedance, fault conditions, battery information such as battery health, battery life, voltage, charge state, charging history if rechargeable, MRI status, Bluetooth connection logs, connection with Clinician Programmer, hours of operation/duration of implant, and the like. Other device information may include device model and lead model.

The neuromodulation device may include communication circuitry 405 configured for use to communicate with other device(s) such as a programming device 211, remote control, phone, tablet and the like. The healthcare-related data may be transferred out from the neuromodulation device for transfer to a data processing system, as discussed above. As shown, a programming device 211 includes a programming control circuit 431, a user interface 432 (e.g., including a user input device 433 such as buttons and a display screen 434), a controller 435, and other components (e.g., an external communication device, not shown) to effect programming of a connected neurostimulation device. The operation of the neurostimulation parameter selection circuit 436 enables selection, modification, and implementation of a particular set of parameters or settings for neurostimulation programming (e.g., via selection of a program, specification by a closed-loop or open-loop programming process, specification by a patient or clinician, or the like).

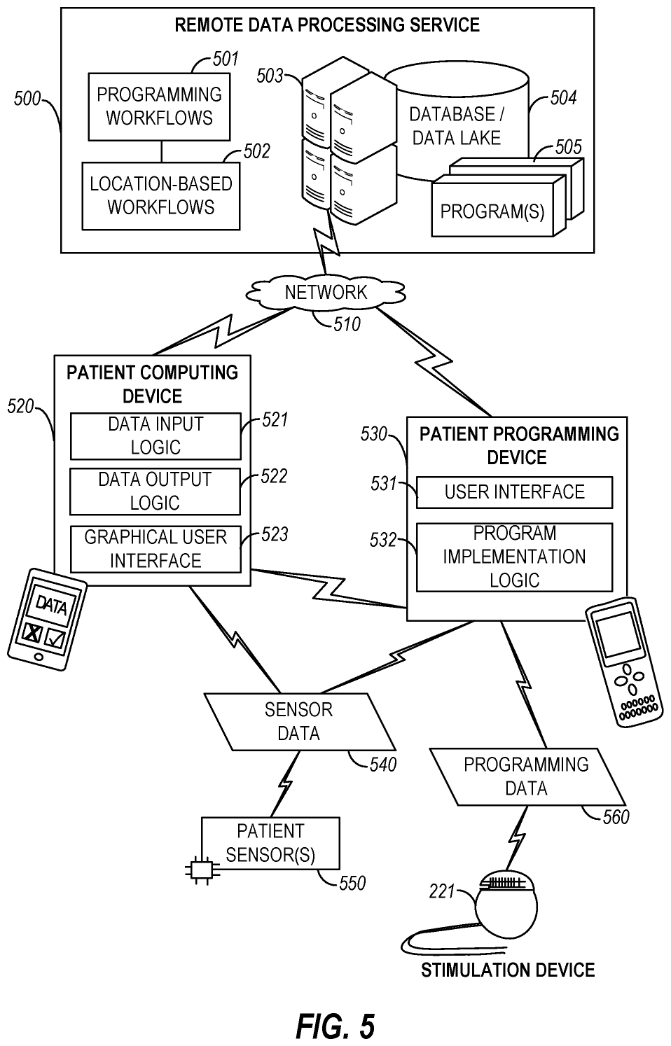

FIG. 5 illustrates, by way of example, an embodiment of data interactions among a data processing system implemented as a remote data processing service 500, for operation of a stimulation device 221 with use of location-assisted programming and location-based workflows. At a high level, a remote data processing service 500 uses algorithms, logic, and other implementations of location-based workflows 502 and neurostimulation programming workflows 501 to collect and analyze patient data collected on an ongoing basis. Such patient data may be hosted in a database 504 or another large-scale data storage (e.g., a data store provided by a data lake) for the patient or a population of patients, and processed by computer hardware 503 of one or more computing systems.

The remote data processing service 500 can be used to perform a variety of data-driven operations, including using the location-based workflows 502 to collect additional event or activity information from the patient (e.g., what activity or outcome occurred at a particular location). The remote data processing service 500 may also use the programming workflows 501 to generate location-driven programming settings and parameters (e.g., in one or more programs 505) that are customized to the human patient. For instance, the remote data processing service 500 may employ the computer hardware 503 as part of the programming workflows 501 or location-based workflows generate or control diagnostic actions, informative alerts or questionnaires, programming recommendations, or programming actions. The programming settings and parameters may be implemented automatically or manually on the stimulation device 221 or via a patient computing device 520 or patient programming device 530.

The remote data processing service 500, in the depicted example, communicates with one or both of the patient computing and programming devices 520, 530, to obtain location and patient data. The remote data processing service 500 may also include data analysis or processing engines (not shown) that parse and determine a state of a particular patient from various inputs and correlate program usage to an activity or location (e.g., to determine what programs and programming settings are beneficial or not beneficial to the patient, when the patient is visiting that location or performing that activity). In some examples, the state of treatment may be based on correlating the historical use of a neurostimulation program or set of parameters with a state of a patient (e.g., identifying that a pain condition became worse or better after beginning use of a particular program) and activities or location characteristics associated with use of a particular program.

The remote data processing service 500 may also analyze a variety of forms of patient input and patient data related to a neurostimulation program or neurostimulation programming parameters. For instance, the remote data processing service 500 may receive information from program usage, questionnaire selections, or even text input originating from a human patient, via the patient computing and programming devices 520, 530. In addition to providing recommended programs, the remote data processing service 500 may provide therapy content and usage recommendations to the patient computing and programming devices 520, 530, including location-based or location-related content.

A patient may enter input data that is related to a location visit, location activities, or other location events via the patient computing device 520 or the patient programming device 530. Additional detail of how input data is collected is discussed with reference to the data processing logic and user interfaces discussed in more detail below. In an example, the patient computing device 520 is a computing device (e.g., personal computer, tablet, smartphone) or other form of user-interactive device that receives and provides interaction with a patient using a graphical user interface 523. The patient computing device 520 may include data input logic 521 and data output logic 522 to control various interaction or location functions. For instance, the data input logic 521 may receive and ask for input from a patient via questionnaires, surveys, messages, or other inputs. The inputs may provide text related to pain or satisfaction, for example, which can be used to identify a psychological or physiological state of the patient, neurostimulation treatment results, or related conditions, including those occurring at a particular location or location type.

A patient programming device 530 is depicted as including a user interface 531 and program implementation logic 532. The program implementation logic 532 specifically may provide the patient with the ability to implement or switch to particular neurostimulation (including those programs 505 generated or updated by remote data processing service 500). The patient programming device 530 may be used for closed-loop programming such as with the receipt of instructions, recommendations, or feedback (including clinician recommendations, behavioral modifications, etc., selected for the patient) that are automatically selected based on detected conditions.

The remote data processing service 500 may also utilize sensor data 540 from one or more patient sensors 550 (e.g., wearables, sleep trackers, motion tracker, implantable devices, etc.) among one or more internal or external devices. The sensor data 540 may be used to determine a customized and current state of the patient condition or neurostimulation treatment results, including those tracked or associated with a particular location or event. In various examples, the stimulation device 221 also includes sensors which contribute to the sensor data 540 to be evaluated by the remote data processing service 500.

In an example, the patient sensors 550 are physiological or biopsychosocial sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

FIG. 6 illustrates data types and processing logic associated with the data input or output examples discussed above for location-based data processing. The data processing logic 610 (e.g., implemented by data processing system 100, remote data processing service 500, or other systems discussed above) may operate to perform data-driven actions and workflows, with the use of geolocation data processing logic 611. Such geolocation data processing logic 611 may be used to trigger particular workflows involving a patient, a clinician, or a caregiver, based on one or more locations or location types, consistent with the examples discussed herein.

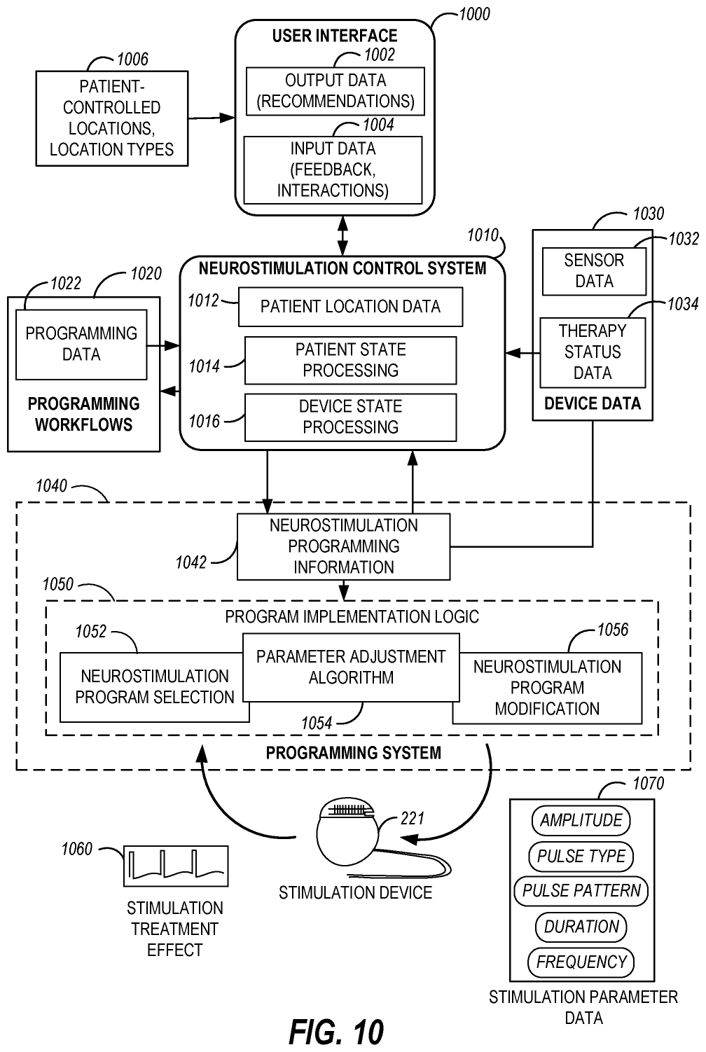

The data processing logic 610 depicts three types of workflows that directly involve the patient. This includes: a workflow for patient reprogramming logic 612, which may enable recommended or automatic device reprogramming based on location activity and events to a location (generally referred to herein as a patient “visit” to a location); a workflow for patient event logic 613, which may cause particular event logging, alerts, notifications, or data analysis based on one or more patient activities or patient states occurring at a location or a location type; a workflow involving patient interaction logic 614, which may provide location-customized questions, recommendations, and other interactive content (inputs and outputs) based on one or more patient activities or patient states occurring at a location. In still further examples, the patient reprogramming logic 612 may enable closed-loop programming. Further details on an implementation of closed-loop programming is provided with reference to FIG. 10, discussed in more detail below.

The data processing logic 610 also depicts: a workflow provided by caregiver workflow logic 615, which may cause various interactions, alerts, or actions with a caregiver (e.g., a family member, personal nurse, trusted friend, etc.) based on the location activity and the patient visit to a location or location type; and, a workflow provided by clinician workflow logic 616, which may cause various interactions, alerts, or actions with a clinician (e.g., doctor, nurse, medical device company representative, pharmacist, or another medical professional) based on the location activity and the patient visit to a location or location type. Various aspects of artificial intelligence, machine learning, and data-driven logic (not depicted) may be implemented in connection with the workflow or location data processing of the data processing logic 610.

FIG. 6 also shows various types of location-based data processing 620, such as data processing that may occur at a user computing device (e.g., patient computing device 520) on an ongoing basis. Such data processing may include the collection of data such as location sensor data 602, and the correlation of the location sensor data 602 to location tags 604 (e.g., with location tags being represented by identifiers of a particular location type). The collection of the location sensor data 602 may be controlled based on privacy settings 603, such as custom settings or permissions on which locations, location types, and location information can be used or shared. Examples of the usage of location tags 604 are provided in more detail in the examples below, which involve the assignment of tagged location types such as “home”, “doctor's office”, “pharmacy”, and the like.

As an example, location tags 604 may be used to reduce data privacy concerns by preventing the collection of detailed, personally identifying locations like GPS coordinates, while still extracting useful insights from user locations. In an example, the geolocation data 601 includes data values from the location tags 604 that are associated with general types of locations such as: general geographical locations; health related locations; large transit locations; individualized (personal) locations. Examples of health-related locations include locations that are associated with per-patient geographies, e.g., hospitals, pharmacies, doctor's offices, clinician programmers. Examples of large transit locations include airports, and rail/bus stations. Examples of individualized (personal) locations include a patient's home, or another patient-frequented (confirmed by the patient), or commonly visited locations (including those driven by activities of daily living (ADLs) or social understanding). Accordingly, geolocation data 601 may be communicated as a tagged data entry (one of the location tags 604) or another data structure, which identifies the patient, the location type, a time spent at the location, and other activity characteristics.

In specific examples, the geolocation data 601 can be used to associate particular characteristics or activities of a patient visit to the type of location, to prevent communicating personally identifying location information from a user (client) device to a data processing (server) system. The location-based data processing 620 also shows the collection and processing of a variety of data types and logic, to determine characteristics or activities occurring at a particular location type. Examples include: Weather event data 621, which is processed by weather event logic 631; Duration event data 622 (e.g., time spent at a location), which is processed by duration event logic 632; Travel or movement event data, which is processed by travel/movement event logic 633; Purpose event data 624, which is processed by purpose event logic 634; Social interaction event data 625, which is processed by social interaction event logic 635. Other related characteristics such as rate of change between locations, visits to new locations or location types for extended time, and other time-based events or properties may also be tracked. Other relevant data fields to be tracked can include analysis of who else is near the patient (e.g., how often and where the patient comes in contact with a caregiver or treatment specialist such as a doctor or medical device company representative).

Still other types of objective data and subjective data may be collected or processed by the logic at the data processing 620, including data that is not directly associated with location. Objective data, as used herein, is data that can be obtained from a measurement or direct observation. Objective data may be measured by a sensor and may be provided via user input when the user has access to objectively determined information. Categories of objective data may include physiological parameter data, therapy data, device data, and environmental data. By way of example and not limitation, physical parameter data may include data such as: heart rate, blood pressure, respiration rate, activity, posture, electromyograms (EMGs), neural responses such as evoked compound action potentials (ecaps), glucose measurements, oxygen levels body temperature, oxygen saturation and gait. By way of example and not limitation, therapy data may include: neuromodulation programs, therapy on/off schedule, dosing, neuromodulation parameters such as waveform, frequency, amplitude, pulse width, period, therapy usage and therapy type. By way of example and not limitation, device data may include: battery information (voltage, charge state, charging history if rechargeable), impedance data, faults, device model, lead models, MRI status, Bluetooth connection logs, and connection history with a Clinician's Programmer (CP). By way of example and not limitation, environmental data may include: temperature, air quality, pressure, location, altitude, sunny, cloudy, precipitation, etc. Subjective data can include information received from the patient or another human user (e.g., caregiver, clinician, etc.). For example, the patient's quantification of pain can provide subjective data. Subjective data may generally involve user-inputted data. Examples of subjective data include questions with free text answers, multiple choice questions, question tree(s), and different question subject(s). Other data may be stored and/or transferred, including detected event data to track events (e.g., that trigger a response, change data resolution), contextual data, time data, and the like. The event(s), context(s) and time may be detected by the system or may be provided via user input.

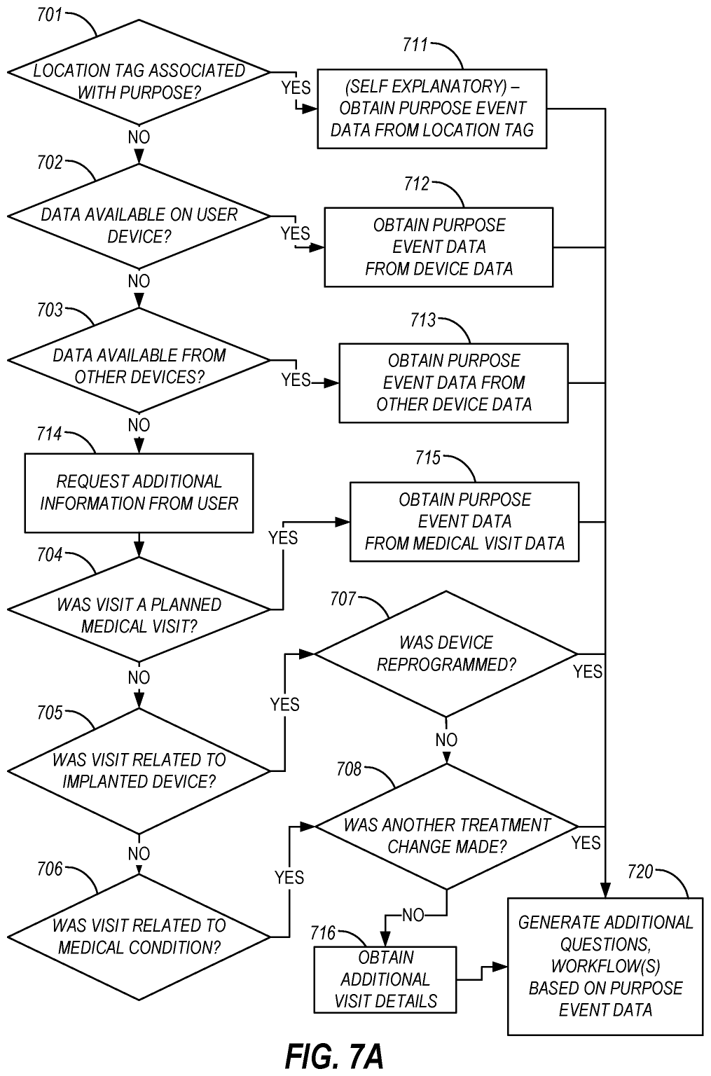

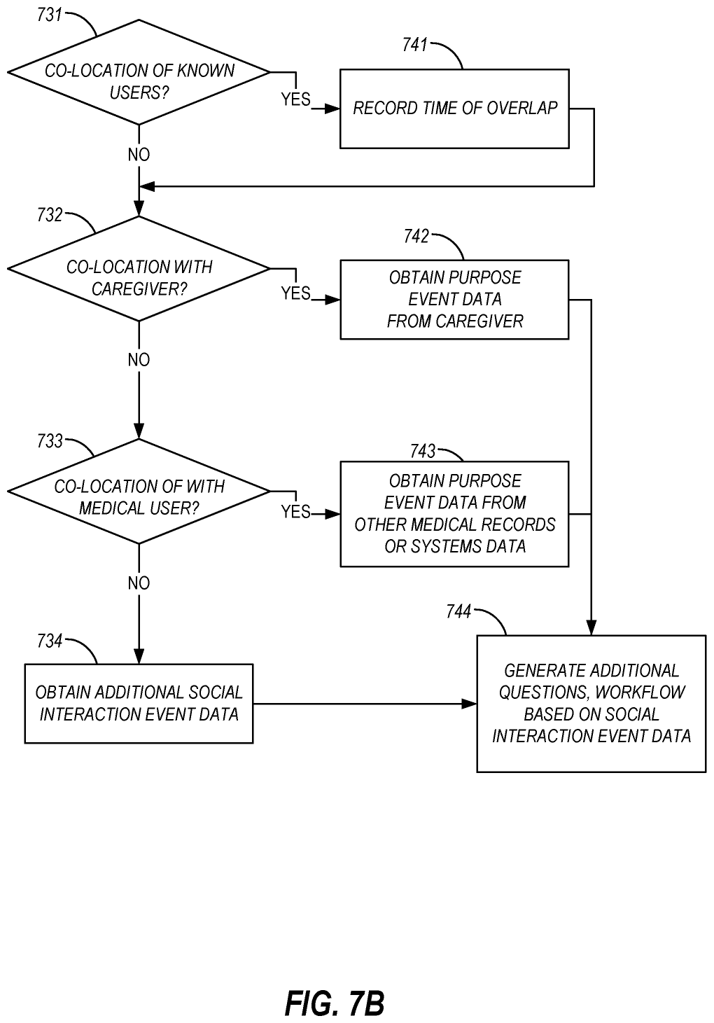

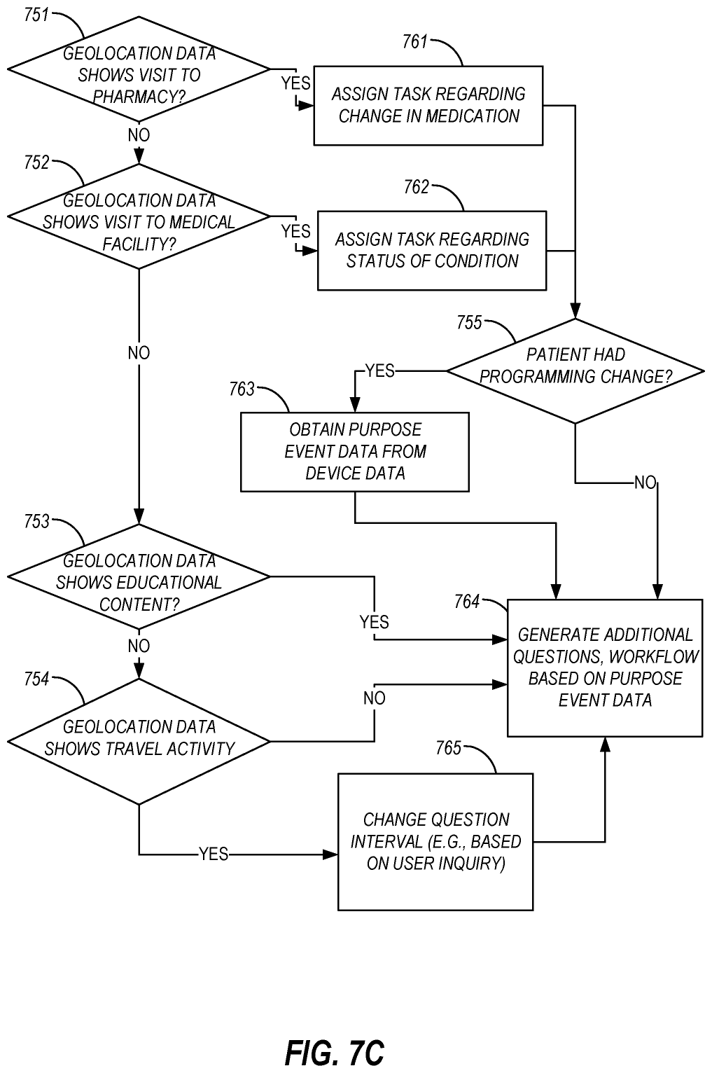

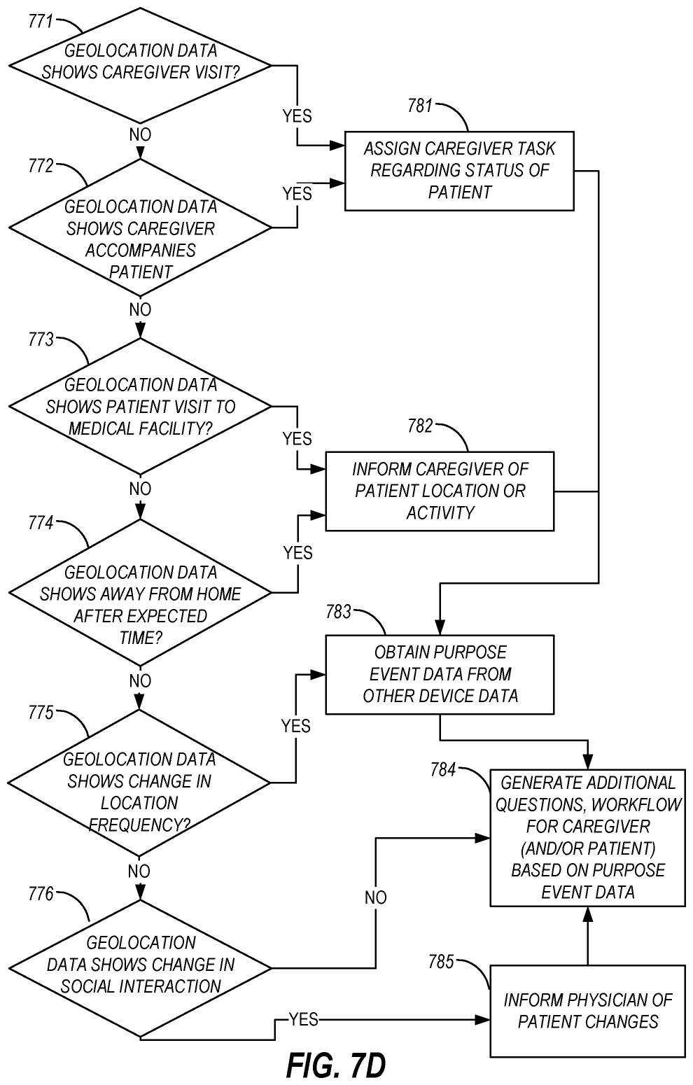

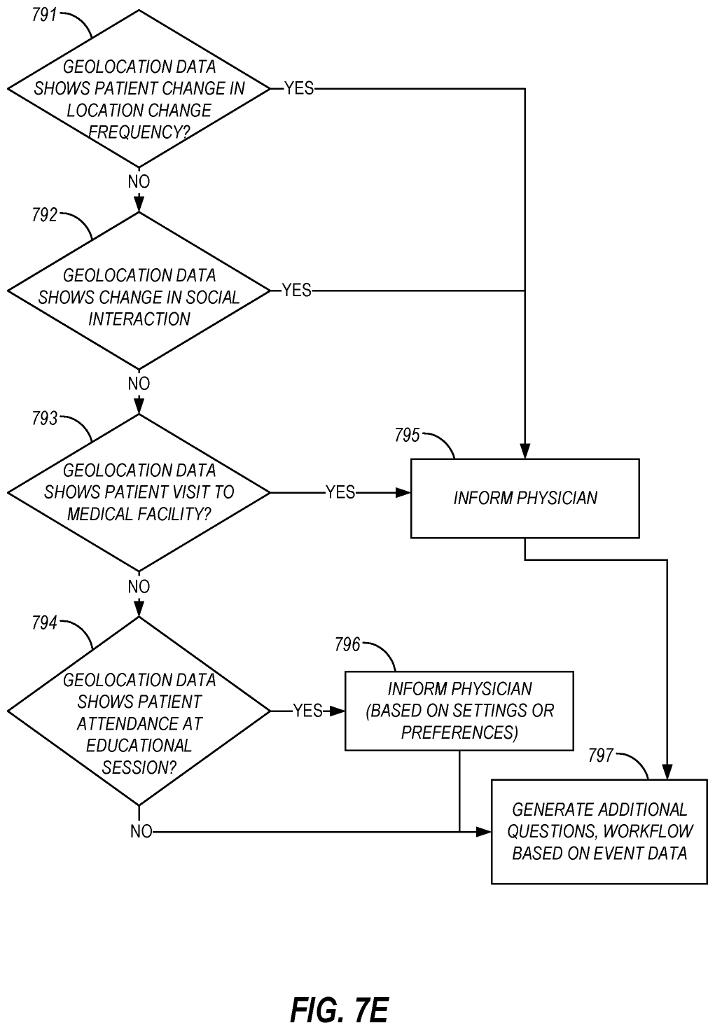

From the above-described data and derived information, and data flows, geolocation data 601 can be used to determine what location types that the user has or has not visited, to then initiate or modify tasks presented to the user. FIGS. 7A to 7E depict flowcharts of various data processing evaluations that trigger specific actions in workflows for patients, clinicians, and caregivers, followed by specific data collection actions, alerts, notifications, recommendations, and the like.

FIG. 7A depicts a flowchart of example workflow operations for deriving purpose of a user visit to a location, such as may be incorporated into purpose event logic 634 and processing of purpose event data 624. Other approaches and workflows may be provided for the processing of purpose event data 624.

At 701, an evaluation is performed to determine if tagged location data (e.g., data of some patient visit to one or more locations or location types) is associated with a purpose. For example, if a purpose of the location visit may be derived from a location title (e.g., home, pharmacy), then the purpose event data is self-explanatory and can be derived from the location tag at 711.

At 702 and 703, an evaluation is performed to determine if additional data is available on the user's computing device or from other devices (e.g., from the neurostimulation device, from a wearable, etc.) to determine a purpose. Location tags such as “hospital” or “doctor's office” may require additional clarification. This may be obtained from purpose event data provided from neurostimulator device data at 712, or from other device data at 713. Data on or associated with the neurostimulator device may include a scheduled appointment with an associated medical provider, an indication of a treatment change (e.g., neurostimulation device reprogramming), and the like. Data associated with other devices or systems may include clinical programmer records, medical device company representative software applications or data entries, and the like.

At 714, a request may be made for additional information from the patient. This may include sending tasks, questionnaires, or other inquiries to a patient to provide more information about what is or was occurring at the given location. For instance, the additional information may be used at 704 to evaluate if a medical visit was planned or scheduled at the location of the visit. If this was a medical visit, then additional purpose event data may be obtained from medical records at 715. If the medical visit was not planned, then information may be used at 705 to evaluate whether the visit is related to the neurostimulation (implanted) device, or at 706 whether the visit is related to the medical condition being treated. Additional evaluations may be performed at 707 to evaluate if the neurostimulation device was reprogrammed, or at 708 to determine if another treatment change was made. Additional medical visit details or data may be obtained at 716. This may be used to generate additional questions or workflows (e.g., based on the purpose event data).

As non-limiting examples, the workflow in FIG. 7A may be used to confirm that clinician programmer changes were made during a visit to a particular clinical location (e.g., tagged as “doctor's office”) as part of a medical visit. Likewise, the workflow in FIG. 7A may be used to determine that a patient was in the same location as a particular medical device representative or physician-associated clinical programmer system. As another example, other treatment changes not directly associated with the device (such as medication changes) may be tracked and identified from this workflow. This may lead to follow-up questions, tracking actions (e.g., looking for a patient visit to a location that is tagged as a pharmacy), and the like.

FIG. 7B depicts a flowchart of example workflow operations for deriving social interaction of a user visit to a location. Such workflow operations may be incorporated into social interaction event logic 635 and processing of social interaction event data 625, and other location-based data processing 620.

At 731, an evaluation is performed to determine if the user being tracked is co-located with other users (e.g., other users of the same software app). If multiple users are located at the same geolocation, then a time of overlap can be logged as in 741. At 732, an evaluation is performed to determine if there is a co-location of two or more users with an established relationship, such as a co-location with a caregiver. In an example, a caregiver software application can identify to a patient software application that the caregiver is located at or near (in proximity to) the patient user. If there is co-location with a caregiver, then additional information such as purpose event data can be obtained from the caregiver as in 742.

At 733, a similar evaluation is performed to determine if there is a co-location of two or more users with an established relationship, such as a co-location with a medical user or medical facility device (e.g., doctor, medical device representative, clinical programmer system, etc.). In an example, the presence of a medical user or system (and particularly multiple of medical users or systems) will indicate a medical setting rather than a social setting, and can be used to derive a patient medical visit. If there is co-location with a medical user or system, then additional information such as purpose event data can be obtained from medical records or systems as in 743. In further examples, co-location of multiple patients as well as medical device company representatives, physicians, or other staff for similar amounts of time and overlap could also indicate an educational event at which multiple users are attending. For instance, the presenter of an educational event could indicate on their software app what information was covered, and the proximity of the patient to this event could automatically track that the patient attended the event and what information (training or education) was covered. If co-location cannot be determined using the flows above, additional social interaction event data can be obtained directly from the user at 734. The social interaction event data and the purpose event data may be used at 744 to generate specific questions or workflow actions based on social interactions that have occurred or should have occurred.

FIG. 7C depicts a flowchart of example workflow operations for targeting tasks to patients based on location data. Such workflow operations may be incorporated into purpose event logic 634 and processing of purpose event data 624, travel/movement event logic 623 and travel/movement event logic 633, and other location-based data processing 620. Here, these workflow operations show how tasks can be sent to patient users based on collected geolocation data and derived information.

At 751, an evaluation is made to determine whether the geolocation data shows that a patient visited a pharmacy. In response, a task is assigned at 762 to query a patient about whether any of the patient medications have changed. At 752, an evaluation is made to determine whether the geolocation data shows that a patient is at a medical facility such as a hospital. In response, a task is assigned at 762 to query a patient about whether the patient is seeing a doctor about a medical condition related to the neurostimulation treatment. Additional suggestions may include suggested agenda questions for the medical visit, or a record of your therapy. For instance, a record of patient data may be provided by access via a QR code (e.g., “Ask your physician to scan this QR code to view the data you have consented to share” or “Ask your physician to scan this QR code to enter the data you have consented to share into your medical record”). At 755, a further evaluation is made to determine whether the geolocation data plus programmer interaction shows that the patient has experienced a neurostimulation device programming change. In response, purpose event data can be obtained at 763 from the device directly, and a task can be assigned to patient at 764, asking how the patient likes the new program.

At 753, an evaluation is made to determine whether the geolocation data can be matched to other data (e.g., device manufacturer representative data) that shows whether the patient attended an educational session. In response, a task may be assigned to the patient at 764, to collect follow-up information (e.g., “Was the session informative?”, “Is there anything you have additional questions about”, “Here are more resources for obtaining more information”).

At 754, an evaluation is made to determine that the geolocation shows travel activity of the patient, such as where a patient location is first detected at an airport and the patient is now located a significant distance away from home. In response, actions may be implemented at 765 to change a question or interaction interval (e.g., to reduce questioning of a patient until return to a home location, or a set time such as two weeks has passed). A system may mark that the patient is likely traveling. If time away occurs without a significant change of location, or the time away exceeds a set time (such as one month), a task can be assigned to the patient to inquire when the patient is traveling, when does the patient expect to return, or to inquire whether the patient has made a permanent relocation.

FIG. 7D depicts a flowchart of example workflow operations for targeting tasks and information to caregivers, based on patient location data. Such workflow operations may be incorporated into location-based data processing 620 and caregiver workflow logic 615. In this example, patients may consent to allow caregivers to view/enter information on their behalf in a separate caregiver software application. Such a software application would be associated with one or more patients linked to the caregiver. The following thus provides various examples of how location-related information can be used to send tasks/information to a caregiver via a caregiver application.

At 771, an evaluation is performed to determine whether the geolocation shows that the caregiver saw the patient today for some period of time qualifying as a visit (e.g., 15 minutes or more). This may also occur at 772 with an evaluation that is performed to determine whether the caregiver accompanies the patient to some location. In response, a task can be assigned to a caregiver at 781 to inquire how was the patient feeling, how was their disease, or did they have any concerns. Such tasks may be limited to days and times when the caregiver has seen the patient. In an example, if a caregiver and patient are together at hospital, pharmacy, educational event, or travel together. Tasks can be prompted to caregiver in addition to the tasks assigned to a patient (as discussed above).

At 773, an evaluation is performed to determine whether the geolocation shows that the patient is located at or has visited a medical facility (e.g., hospital), and optionally is not near a clinical programmer or medical device manufacturer representative and did not have a scheduled visit. In response, the caregiver can be informed at 782 of the patient's location and the ongoing activity. Similarly, at 774, an evaluation is performed to determine whether the geolocation shows that the patient is located away from home (and has not returned home) after an expected time. In response, the caregiver can be informed at 782 of the patient's activity.

At 775, an evaluation is performed to determine whether the geolocation shows that the patient has a change in location frequency, such as when the patient has left home more or less than usual. In response, additional purpose event data can be obtained as in 783 from a device to identify whether there is associated medical data showing whether the patient is feeling sick. This can be followed at 784 by additional questions or workflows for the caregiver and/or patient based on the purpose event data and the detected changes.

At 776, an evaluation is performed to determine whether the geolocation shows that the patient has increased or decreased their social interactions. In response, a clinician such as an overseeing physician can be informed of the changes such as at 785. Additional questions or workflows for the caregiver and/or patient can also be triggered based on the changes.

FIG. 7E depicts a flowchart of example workflow operations for informing physicians or other medical professionals based on patient location data. Such workflow operations may be incorporated into location-based data processing 620 and clinician workflow logic 616. While physicians may not be directly sent tasks like patients and caregivers, information derived from the geolocation data can be provided to physicians and used for additional workflows and follow-up actions.

At 791, an evaluation is performed to determine whether the geolocation data shows that patient has left home more/less than usual. Or, at 792, an evaluation is performed to determine whether the geolocation data shows that patient has increased or decreased their social interactions. Or, at 793, an evaluation is performed to determine whether the geolocation data (or, a patient caregiver confirmation) indicates that the patient is/was in the hospital, with an unscheduled visit. In response to any of these scenarios, the physician may be informed of the event as in 795. Additional questions and workflow actions at 797 may also be provided by on these events and other event data.

At 791, an evaluation is performed to determine whether the geolocation data plus some other data (e.g., representative data) shows that a patient visited an educational session. In response, at 796, a physician may be informed of the event (e.g., based on settings or preferences). Additional questions and workflow actions at 797 may also be provided by on these events and other event data.

Various approaches may be used to deidentify location data while capturing significant characteristics about a patient visit to a particular location. In an example, GPS coordinates and personally identifying location information is maintained only on the user's personal device, and the user's personal device will only transmit tags and location type characteristics to a processing system. In one example, a patient operates a software application (e.g., on the patient's smartphone) that will identify the user's latitude and longitude at all times (upon consent). A list of tagged locations including the location coordinates will be downloaded to the patient's phone and stored as a list. As a user travels to different location, the application will compare the current GPS settings to the list of known locations, and will register new locations as applicable.

FIG. 8A and FIG. 8B depict data input user interfaces 810A, 810B (e.g., a smartphone app graphical user interface) which provide output on a user device 800 (e.g., a smartphone). In various examples, a patient's phone, computer, tablet, or programming remote control (or, multiple of these devices) may provide the illustrated user interfaces. The user interfaces 810A, 810B are also depicted to include graphical touchscreen interactivity, but it will be understood that other user input functionality, such as menu selection functionality 801, voice input functionality 802, or text input functionality 803 (including non-graphical inputs such as voice interactions controlled by a chatbot or by virtual assistants or agents such as Amazon® Alexa, Google® Assistant, Apple® Siri, etc.) may be provided.

FIG. 8A depicts the data input user interface 810A as including functionality to enable tagging of locations and location types. For example, the user device 800 may maintain a “map” of pre-tagged locations and track visits to these locations in an ongoing basis. When a user's location is within a certain proximity of pre-tagged locations for a set length of time and at a set acceleration (walking or slower), the user device 800 will determine that the patient is located at a pre-tagged location. The patient will be tagged as being located at this location. At a later time, the user device 800 will transmit the tag as well as information such as what times the patient was at the location, how long the patient was at the location, etc. to the data processing system.

FIG. 8B similarly depicts the data input user interface 810B as including functionality to enable tagging of a visit to an un-tagged location. For example, when a user visits an un-tagged location for a set length of time at a set acceleration (walking or slower), the location will be assigned a random tag until more precise information can be determined. The data input user interface 810B can be used to select a location type or activity type of the location. For un-tagged locations, various thresholds can be established (e.g., how often the user visits the location, how much time (duration) the user spends at the location, and whether other users are present). In some examples, if un-tagged locations are visited for long periods of time, are visited frequently, or have multiple users present, then the un-tagged location can be presented with an elevated priority for receiving a tag.

Various user interface features may be provided for editing, adding, and removing location tags (and, for allowing consent to the use of such location tags). As an example, the user must consent to allow the software app to have access to the phone's GPS data. Information can be provided to a user explaining that the GPS data resides locally (on the user's smartphone) and only non-specific (non-identifying) patient location tags are shared.

Functionality may be provided to enable a user to choose to manually edit, add or remove a tag at any time. When a user removes a tag, that tag may remain on the personal device but have an additional variable to indicate that the user does not wish to share this location. When the patient passes through the location, the app will recognize it but will not save additional data and will not transmit location data. In another implementation, the tag may be fully removed from the device and any databases if removed by a user.

In a specific example, any time that a user edits/adds a tag to a location, a prepopulated list can be presented as a dropdown, multi-select or a similar format. In still further examples, when a location tag of “other” is selected, the user will be given a free text entry option to self-define the tag. This free text entry data can be transmitted as written or subject to free text formatting, and may include filters meant to remove identifying features such as names of specific medical facilities or persons.

FIG. 9A and FIG. 9B depict output data user interfaces 910A, 910B which provide various examples of outputting data and interactions based on location activity, such as within an app executing on the smartphone mobile device 800. Additional menus and user interface functionality are not shown for purposes of simplicity.

FIG. 9A depicts the data input user interface 910A as including functionality to receive further information regarding the patient state and patient activity at a particular location. For instance, a chatbot may provide questions 921, 923 which are customized to the patient's response 922 and location activity (e.g., a visit to a location type tagged “Neighborhood Park”). FIG. 9B also depicts another example of the data input user interface 910B as including information 931 to inform a user or caregiver of activity changes, and user inputs 932 to input a choice in response to a questionnaire. Other patient interaction workflow questions and actions may be provided based on location activity and characteristics of a patient visit to a particular location or location type.

FIG. 10 illustrates, by way of example, an embodiment of a data processing flow for controlling the neurostimulation treatment of a human patient, which may occur based on the location-based data processing operations discussed above. Specifically, this data processing flow shows how a neurostimulation control system 1010 may perform patient state processing 1014 and device state processing 1016 functions, based on patient location data 1012 (discussed above) that indicates activities or events of the patient or another related party at one or more location. Here, as the patient state processing 1014 produces a patient state and the device state processing 1016 produces a device state, various improvements to neurostimulation programming can be provided (e.g., with programming workflows 1020), which in turn produces updated programming data 1022 for deployment with new neurostimulation programming information 1042).

In this example, the user interface 1000 provides output data 1002 (e.g., recommendations, questions, etc.) to help collect location-related data on the activity of the patient and the usage of neurostimulation programs. The user interface 1000 collects input data 1004 (e.g., feedback data, commands, other user interactions), as discussed above, determine patient locations and location types, including visits to particular locations that occur during the use of the one or more neurostimulation programs. Such operations and functionality may be consistent with the user interfaces discussed above, although other data inputs and outputs may be used.

FIG. 10 also depicts the evaluation of device data 1030, such as sensor data 1032, therapy status data 1034, and other treatment aspects which may be obtained or derived from the stimulation device 221 or related neurostimulation programming and device operation. The device data 1030 and the inputs received with the user interface 1000 allow a set of patient location data 1012 to be collected, which can be evaluated in addition to patient state processing functions 1014 and device state processing functions 1016. Various programming workflows 1020 generates new or updated programming data 1022 (e.g., programs, program parameter values) based on some combination of the patient location data 1012, and from results of the patient state processing functions 1014 and device state processing functions 1016.

The remainder of the data processing flow illustrates how the neurostimulation control system 1010 implements programming, such as in a closed loop (or partially-closed-loop) system. A programming system 1040 uses programing information 1042 provided from the neurostimulation control system 1010 as an input to program implementation logic 1050. This may be affected, in part, by device data 1030 including sensor data 1032 and therapy status data 1034. The program implementation logic 1050 may be implemented by a parameter adjustment algorithm 1054, which affects a neurostimulation program selection 1052 or a neurostimulation program modification 1056. For instance, some parameter changes may be implemented by a simple modification to a program operation; other parameter changes may require a new program to be deployed. The results of the parameter or program changes or selection provides various stimulation parameters 1070 to the stimulation device 221, causing a different or new stimulation treatment effect 1060.

By way of example, operational parameters of the stimulation device which may be generated, identified, or evaluated by the neurostimulation control system 1010 may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output, and apply any number of control techniques to modify the electrical simulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

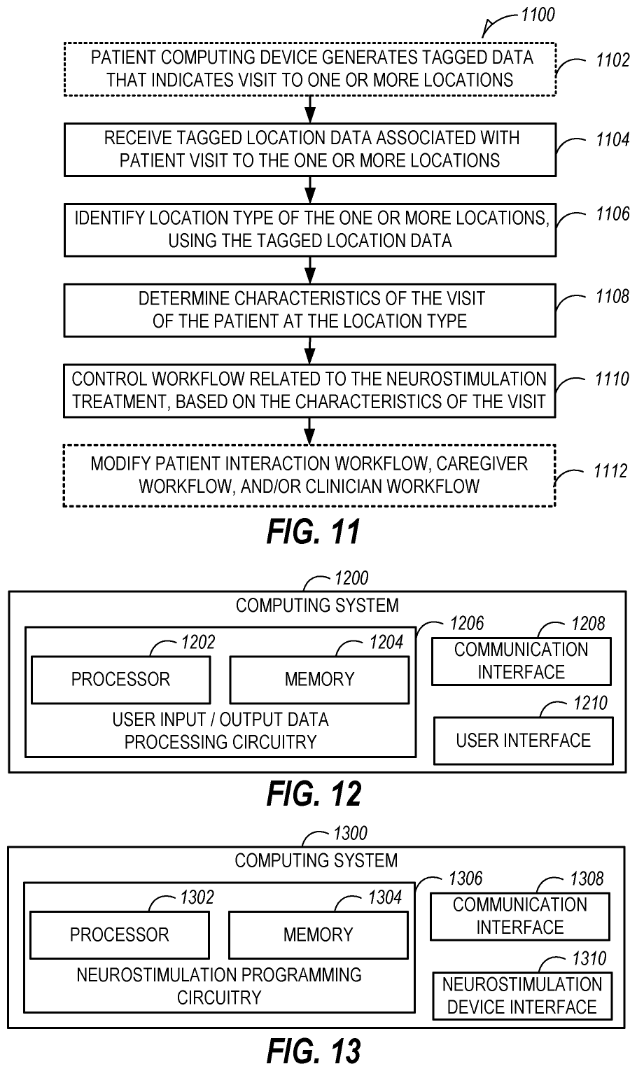

FIG. 11 illustrates, by way of example, an embodiment of a processing method 1100 implemented by a system or device to analyze patient location activity in connection with a neurostimulation treatment programming, e.g., in connection with location data collected herein. For example, the processing method 1100 can be embodied by electronic operations performed by one or more computing systems or devices (including those at a network-accessible remote service) that are specially programmed to implement data analysis and/or neurostimulation data processing operations. In specific examples, the operations of the method 1100 may be implemented through the systems and data flows depicted above, at a single entity or at multiple locations.

In an example, the method 1100 begins at 1102 by the use of a patient computing device, to generate tagged location data that tracks and indicates a visit by the patient (i.e., the patient who is undergoing the neurostimulation treatment) to one or more locations. The tagged location data can include an identifier of the location type, such as in a scenario where the location type is one of a plurality of defined location types. The tagged location data can also include an identifier associated with the patient or the patient computing device. Other information may be tracked and provided in the tagged location data, consistent with the examples above.

The method 1100 continues at 1104 by receiving tagged location data associated with the patient visit to the one or more locations. In various examples, the tagged location data is directly or indirectly provided from a patient computing device. The identifier of the location type (and other aspects of the tagged location data) may be automatically provided by the patient computing device, in response to the patient computing device having detected the visit of the patient to one or more geographic locations associated with the location type. In some examples, the tagged location data provided from the patient computing device includes only information regarding the location type, and does not include an identifiable geographic location of the one or more locations.

The method 1100 continues at 1106 by identifying a location type of the one or more locations, using the tagged location data. In an example, the location type is a type of medical care location, such as for a location type that is categorized as one (or more) of a: hospital, clinic, rehabilitation facility, nursing home facility, programming location, pharmacy, or other medical-related facility. The location type may be associated with one or more medical professional or service in connection with the neurostimulation treatment for the patient or for others. In other examples, other medical or health-related locations (e.g., dialysis centers, doctor offices) may be tracked, such as to track medical locations that are not directly associated with the medical condition being treated by the neurostimulation. The association of a location type with a particular tracked location may be under the control by the patient. For instance, an identifier of the location type may be provided by the patient computing device, in response to (after) selection of the location type by a user of the patient computing device.

The method 1100 continues at 1108 by determining the characteristics of the visit of the patient at the location type. In an example, the characteristics of the visit of the patient to the location type are determined from one or more of: weather event data associated with weather at the one or more locations; duration event data associated with a duration of the visit at the one or more locations; travel event data associated with travel of the patient to or from the one or more locations; purpose event data associated with a purpose of the visit at the one or more locations; or social interaction event data associated with interactions occurring between the patient and one or more persons at the one or more locations.

The method 1100 continues at 1110 by controlling a workflow related to the neurostimulation treatment, based on the determined characteristics of the visit. In an example, this treatment workflow is based on determining usage of a neurostimulation program by a neurostimulation device at the location type, and identifying a patient state based on the usage of the neurostimulation program and the characteristics of the visit of the patient at the location type. For instance, the treatment workflow may provide a control to a closed-loop programming therapy of the neurostimulation device based on the identified patient state. This identified patient state can be related to one or more of: sleep, actigraphy, accelerometry, pain, movement, stress, disease-related symptoms, emotional state, medication state, or activity during use of the neurostimulation program. In a specific example, the use of a closed-loop programming therapy causes an automatic change to neurostimulation programming settings on the neurostimulation device, as the automatic change to the neurostimulation programming settings controls one or more of: pulse patterns, pulse shapes, a spatial location of pulses, electric fields or activating functions of active electrodes, waveform shapes, or a spatial location of waveform shapes, for modulated energy provided with a plurality of leads of the neurostimulation device.

In further examples, the method 1100 continues at 1112 by optionally implementing or modifying a patient interaction workflow, a caregiver workflow, and/or a clinician workflow. In an example, the workflow related to the neurostimulation treatment is a patient interaction workflow to occur with the patient, and the patient interaction workflow causes the generation of one or more questionnaires or interaction tasks to provide to the patient (e.g., based on the characteristics of the visit of the patient at the location type). In another example, the workflow related to the neurostimulation treatment is a caregiver workflow to occur with a caregiver or a medical device company representative associated with the patient, and the caregiver workflow cause generation of one or more alerts to at least one of the caregiver or a medical device company representative (e.g., based on the characteristics of the visit of the patient at the location type). In still another example, the workflow related to the neurostimulation treatment is a clinician workflow to occur with a clinician or a medical device company representative associated with the patient, and the clinician workflow causes the generation of one or more alerts to at least one of the clinician or the medical device company representative (e.g., based on the characteristics of the visit of the patient at the location type).

FIG. 12 illustrates, by way of example, a block diagram of an embodiment of a system 1200 (e.g., a computing system) for performing analysis of patient location data (e.g., tagged location data collected with use of a user interface 1210) in connection with the data processing operations discussed above. The system 1200 may be integrated with or coupled to a computing device, a remote control device, patient programmer device, clinician programmer device, program modeling system, or other external device, deployed with neurostimulation treatment. In some examples, the system 1200 may be a networked device (server) connected via a network (or combination of networks) which communicates to one or more devices (clients) using a communication interface 1208 (e.g., communication hardware which implements software network interfaces and services). The network may include local, short-range, or long-range networks, such as Bluetooth, cellular, IEEE 802.11 (Wi-Fi), or other wired or wireless networks.

The system 1200 includes a processor 1202 and a memory 1204, which can be optionally included as part of user input/output data processing circuitry 1206. The processor 1202 may be any single processor or group of processors that act cooperatively. The memory 1204 may be any type of memory, including volatile or non-volatile memory. The memory 1204 may include instructions, which when executed by the processor 1202, cause the processor 1202 to implement data processing, or to enable other features of the user input/output data processing circuitry 1206. Thus, electronic operations in the system 1200 may be performed by the processor 1202 or the circuitry 1206.

For example, the processor 1202 or circuitry 1206 may implement any of the user-based features of the method 1100 to obtain and process patient location activity, to generate user interface displays, and to provide tagged data for patient location visits. It will be understood that the processor 1202 or circuitry 1206 may also implement aspects of the logic and processing described above, for use in various forms of closed-loop and partially-closed-loop device programming or related device actions.

FIG. 13 illustrates, by way of example, a block diagram of an embodiment of a system 1300 (e.g., a computing system) implementing neurostimulation programming circuitry 1306 to cause programming of an implantable electrical neurostimulation device, for accomplishing the therapy objectives in a human subject based on neurostimulation programming as discussed herein. The system 1300 may be operated by a clinician, a patient, a caregiver, a medical facility, a research institution, a medical device manufacturer or distributor, and embodied in a number of different computing platforms. The system 1300 may be a remote control device, patient programmer device, program modeling system, or other external device, including a regulated device used to directly implement programming commands and modification with a neurostimulation device. In some examples, the system 1300 may be a networked device connected via a network (or combination of networks) to a computing system operating a user interface computing system using a communication interface 1308. The network may include local, short-range, or long-range networks, such as Bluetooth, cellular, IEEE 802.11 (Wi-Fi), or other wired or wireless networks.

The system 1300 includes a processor 1302 and a memory 1304, which can be optionally included as part of neurostimulation programming circuitry 1306. The processor 1302 may be any single processor or group of processors that act cooperatively. The memory 1304 may be any type of memory, including volatile or non-volatile memory. The memory 1304 may include instructions, which when executed by the processor 1302, cause the processor 1302 to implement the features of the neurostimulation programming circuitry 1306. Thus, the electronic operations in the system 1300 may be performed by the processor 1302 or the circuitry 1306.

The processor 1302 or circuitry 1306 may directly or indirectly implement neurostimulation operations associated with the method 1100, including the use of neurostimulation device programming based on location-modified workflows (operation 1112). The processor 1302 or circuitry 1306 may further provide data and commands to assist the processing and implementation of the programming using communication interface 1308 or a neurostimulation device interface 1310. It will be understood that the processor 1302 or circuitry 1306 may also implement other aspects of the device data processing or device programming functionality described above.

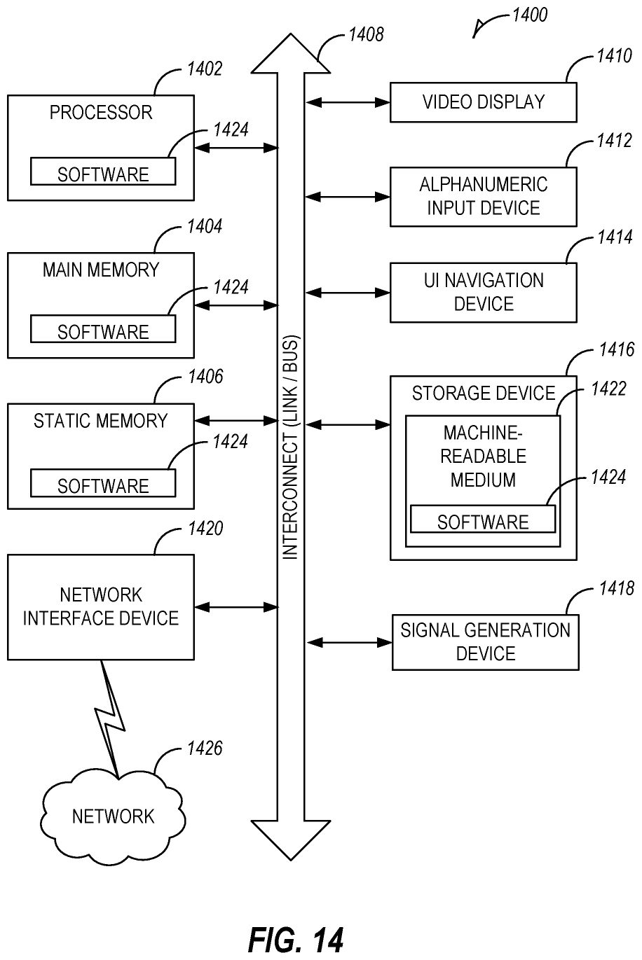

FIG. 14 is a block diagram illustrating a machine in the example form of a computer system 1400, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1400 includes at least one processor 1402 (e.g., a central processing unit (CPU), a graphics processing unit (GPU) or both, processor cores, compute nodes, etc.), a main memory 1404 and a static memory 1406, which communicate with each other via a link 1408 (e.g., bus). The computer system 1400 may further include a video display unit 1410, an alphanumeric input device 1412 (e.g., a keyboard), and a user interface (UI) navigation device 1414 (e.g., a mouse). In one embodiment, the video display unit 1410, input device 1412 and UI navigation device 1414 are incorporated into a touch screen display. The computer system 1400 may additionally include a storage device 1416 (e.g., a drive unit), a signal generation device 1418 (e.g., a speaker), a network interface device 1420, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 14 (such as a GPU, video display unit, keyboard, etc.).

The storage device 1416 includes a machine-readable medium 1422 on which is stored one or more sets of data structures and instructions 1424 (e.g., software) embodying or utilized by any one or more of the methodologies or functions described herein. The instructions 1424 may also reside, completely or at least partially, within the main memory 1404, static memory 1406, and/or within the processor 1402 during execution thereof by the computer system 1400, with the main memory 1404, static memory 1406, and the processor 1402 also constituting machine-readable media.

While the machine-readable medium 1422 is illustrated in an example embodiment to be a single medium, the term “machine-readable medium” may include a single medium or multiple media (e.g., a centralized or distributed database, and/or associated caches and servers) that store the one or more instructions 1424. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1424 may further be transmitted or received over a communications network 1426 using a transmission medium via the network interface device 1420 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A device to analyze patient location activity in connection with a neurostimulation treatment, the device comprising:
one or more processors; and
one or more memory devices comprising instructions, which when executed by the one or more processors, cause the one or more processors to:
receive tagged location data associated with a patient undergoing the neurostimulation treatment, wherein the tagged location data is provided from a patient computing device, wherein the tagged location data indicates a visit of the patient to one or more locations;
identify a location type of the one or more locations, using the tagged location data;
determine characteristics of the visit of the patient at the location type, using the tagged location data; and
control a workflow related to the neurostimulation treatment, based on the characteristics of the visit of the patient at the location type.

2. The device of claim 1, wherein the tagged location data includes an identifier of the location type, wherein the location type is one of a plurality of defined location types, and wherein the tagged location data provided from the patient computing device does not include an identifiable geographic location of the one or more locations.

3. The device of claim 2, wherein the identifier of the location type is automatically provided by the patient computing device, in response to the patient computing device having detected the visit of the patient to one or more geographic locations associated with the location type.

4. The device of claim 3, wherein the location type is a type of medical care location, wherein the location type is categorized as one of a: hospital, clinic, rehabilitation facility, nursing home facility, programming location, or pharmacy, associated with one or more medical professional or service in connection with the neurostimulation treatment; and
wherein the identifier of the location type is provided by the patient computing device, in response to selection of the location type by a user of the patient computing device.

5. The device of claim 2, wherein the tagged location data includes an identifier associated with the patient or the patient computing device.

6. The device of claim 1, wherein the workflow related to the neurostimulation treatment is a patient interaction workflow to occur with the patient, and wherein the instructions further cause the one or more processors to:
generate one or more questionnaires or interaction tasks to provide to the patient based on the characteristics of the visit of the patient at the location type.

7. The device of claim 1, wherein the workflow related to the neurostimulation treatment is a caregiver workflow to occur with a caregiver or a medical device company representative associated with the patient, and wherein the instructions further cause the one or more processors to:
generate one or more alerts to at least one of the caregiver or a medical device company representative based on the characteristics of the visit of the patient at the location type.

8. The device of claim 1, wherein the workflow related to the neurostimulation treatment is a clinician workflow to occur with a clinician or a medical device company representative associated with the patient, and wherein the instructions further cause the one or more processors to:
generate one or more alerts to at least one of the clinician or the medical device company representative based on the characteristics of the visit of the patient at the location type.

9. The device of claim 1, wherein the characteristics of the visit of the patient are determined from one or more of:
weather event data associated with weather at the one or more locations;
duration event data associated with a duration of the visit at the one or more locations;
travel event data associated with travel of the patient to or from the one or more locations;
purpose event data associated with a purpose of the visit at the one or more locations; or
social interaction event data associated with interactions occurring between the patient and one or more persons at the one or more locations.

10. The device of claim 1, wherein the instructions further cause the one or more processors to:
determine usage of a neurostimulation program by a neurostimulation device at the location type; and
identify a patient state based on the usage of the neurostimulation program and the characteristics of the visit of the patient at the location type;
wherein the workflow related to the neurostimulation treatment provides a control to a closed-loop programming therapy of the neurostimulation device based on the identified patient state, and
wherein the patient state relates to one or more of: sleep, actigraphy, accelerometry, pain, movement, stress, disease-related symptoms, emotional state, medication state, or activity during use of the neurostimulation program.

11. A method for analyzing patient location activity in connection with a neurostimulation treatment, comprising:
receiving tagged location data associated with a patient undergoing the neurostimulation treatment, wherein the tagged location data is provided from a patient computing device, wherein the tagged location data indicates a visit of the patient to one or more locations;
identifying a location type of the one or more locations, using the tagged location data;
determining characteristics of the visit of the patient at the location type, using the tagged location data; and
controlling a workflow related to the neurostimulation treatment, based on the characteristics of the visit of the patient at the location type.

12. The method of claim 11, wherein the tagged location data includes an identifier of the location type, wherein the location type is one of a plurality of defined location types, and wherein the tagged location data provided from the patient computing device does not include an identifiable geographic location of the one or more locations.

13. The method of claim 12, wherein the identifier of the location type is automatically provided by the patient computing device, in response to the patient computing device having detected the visit of the patient to one or more geographic locations associated with the location type.

14. The method of claim 13, wherein the location type is a type of medical care location, wherein the location type is categorized as one of a: hospital, clinic, rehabilitation facility, nursing home facility, programming location, or pharmacy, associated with one or more medical professional or service in connection with the neurostimulation treatment; and
wherein the identifier of the location type is provided by the patient computing device, in response to selection of the location type by a user of the patient computing device.

15. The method of claim 12, wherein the tagged location data includes an identifier associated with the patient or the patient computing device.

16. The method of claim 11, wherein the workflow related to the neurostimulation treatment is a patient interaction workflow to occur with the patient, and wherein the method further comprises:
generating one or more questionnaires or interaction tasks to provide to the patient based on the characteristics of the visit of the patient at the location type.

17. The method of claim 11, wherein the workflow related to the neurostimulation treatment is a caregiver workflow to occur with a caregiver or a medical device company representative associated with the patient, and wherein the method further comprises:
generating one or more alerts to at least one of the caregiver or a medical device company representative based on the characteristics of the visit of the patient at the location type.

18. The method of claim 11, wherein the workflow related to the neurostimulation treatment is a clinician workflow to occur with a clinician or a medical device company representative associated with the patient, and wherein the method further comprises:
generating one or more alerts to at least one of the clinician or the medical device company representative based on the characteristics of the visit of the patient at the location type.

19. The method of claim 11, wherein the characteristics of the visit of the patient are determined from one or more of:
weather event data associated with weather at the one or more locations;
duration event data associated with a duration of the visit at the one or more locations;
travel event data associated with travel of the patient to or from the one or more locations;
purpose event data associated with a purpose of the visit at the one or more locations; or
social interaction event data associated with interactions occurring between the patient and one or more persons at the one or more locations.

20. The method of claim 11, further comprising:
determining usage of a neurostimulation program by a neurostimulation device at the location type; and
identifying a patient state based on the usage of the neurostimulation program and the characteristics of the visit of the patient at the location type;
wherein the workflow related to the neurostimulation treatment provides a control to a closed-loop programming therapy of the neurostimulation device based on the identified patient state, and
wherein the patient state relates to one or more of: sleep, actigraphy, accelerometry, pain, movement, stress, disease-related symptoms, emotional state, medication state, or activity during use of the neurostimulation program.
